(19) United States
(12) Patent Application Publication
(10) Pub. No.: US 2010/0298347 A1
(43) Pub. Date: Nov. 25, 2010

(54) SUBSTITUTED 2-NAPHTHOIC ACIDS AS ANTAGONISTS OF GPR105 ACTIVITY

(75) Inventors: Michel Belley, Pierrefonds (CA); Denis Deschenes, Dorval (CA); Rejean Fortin, Montreal (CA); Jean-Francois Fournier, Montreal (CA); Sebastien Gagne, Montreal (CA); Yves Gareau, Notre-Dame de l'ile-Perrot (CA); Jacques Yves Gauthier, Laval (CA); Lianhai Li, Pierrefonds (CA); Joel Robichaud, Dollard-des-Orrneaux (CA); Michel Therien, Laval (CA); Geoffrey K. Tranmer, Westmount (CA); Zhaoyin Wang, Kirkland (CA)

Correspondence Address: MERCK, P O BOX 2000, RAHWAY, NJ, 07065-0907, US

(73) Assignee: MERCK FROSST CANADA LTD, Kirkland, QC (CA)

(21) Appl. No.: 12/743,610

(22) PCT Filed: Dec. 1, 2008
(86) PCT No.: PCT/CA2008/002105

§ 371 Date: May 19, 2010

Related U.S. Application Data
(60) Provisional application No. 61/005,266, filed on Dec. 4, 2007.

(51) Int. Cl. A61K 31/519; C07C 63/72; C07D 333/34; C07D 333/22; C07C 63/42; C07D 317/54; C07D 409/12; C07D 487/04; C07D 249/08; C07D 211/94; C07D 211/32; A61K 31/192; A61K 31/381; A61K 31/194; A61K 31/36; A61K 31/4436; A61K 31/4196; A61K 31/451; A61P 3/10; A61P 5/50; A61P 3/04; A61P 3/00

(52) U.S. Cl. 514/259.1; 562/467; 549/66; 562/427; 549/79; 562/488; 549/447; 546/280.4; 544/281; 548/268.6; 546/242; 546/248; 514/569; 514/445; 514/438; 514/464; 514/336; 514/383; 514/319

(57) ABSTRACT

Substituted 2-naphthoic acids of structural formula are effective as antagonists of the biological activity of GPR105 protein. They are useful for the treatment, control or prevention of disorders responsive to antagonism of this receptor, such as diabetes, particularly, Type 2 diabetes, insulin resistance, hyperglycemia, lipid disorders, obesity, atherosclerosis, and conditions associated with the Metabolic Syndrome.

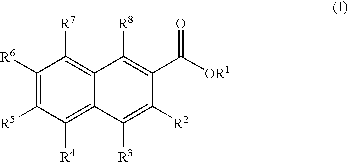

### FIELD OF THE INVENTION

The present invention relates to substituted 2-naphthoic acids which are antagonists of the biological activity of the GPR105 protein and the use of such compounds to control, prevent and/or treat conditions or diseases mediated by the GPR105 protein. The compounds of the present invention are useful for the treatment of diabetes, particularly Type 2 diabetes, hyperglycemia, insulin resistance, lipid disorders, obesity, atherosclerosis, and other conditions associated with the Metabolic Syndrome.

### BACKGROUND OF THE INVENTION

Metabolic Syndrome is a disorder that includes obesity, dyslipidemia, and hyperglycemia. Metabolic Syndrome has increased to epidemic proportions worldwide. The pathophysiology of this syndrome is attributed to central distributed obesity, decreased high density lipoprotein, elevated triglycerides, elevated blood pressure and hyperglycemia. People suffering from Metabolic Syndrome are at increased risk of developing Type 2 diabetes, coronary heart disease, and other diseases related to plaque accumulation in artery walls (e.g., stroke and peripheral vascular disease). In two prospective European studies, Metabolic Syndrome was a predictor of increased cardiovascular disease and mortality (Isomaa et al., “Cardiovascular Morbidity and Mortality Associated With the Metabolic Syndrome,” Diabetes Care 24:683-689, 2001; Lakka et al., “The Metabolic Syndrome and Total and Cardiovascular Disease Mortality in Middle Aged Men,” JAMA 288:2709-2716, 2002).

The most significant underlying cause of Metabolic Syndrome is obesity. It has been disclosed in US 2007/0092913 (published on Apr. 26, 2007) that expression of GPR105 protein is correlated with weight gain and development of Type 2 diabetes. Furthermore, it has been demonstrated that antisense inhibition of GPR105 expression in mice reduces the rate at which the mice gain weight in response to a high fat diet. The mice also have lower levels of insulin, suggesting a decreased level of insulin resistance in these mice. Accordingly, GPR105 is a potential target for drugs that control, prevent, or treat Type 2 diabetes and/or obesity or that ameliorate at least one symptom associated with the Metabolic Syndrome.

The present invention provides a novel class of substituted beta-naphthoic acids as GPR105 antagonists which are useful for the control, prevention, or treatment of obesity and diabetes, in particular, Type 2 diabetes and to ameliorate the symptoms associated with the Metabolic Syndrome.

### SUMMARY OF THE INVENTION

The present invention relates to substituted 2-naphthoic acids of structural formula I:

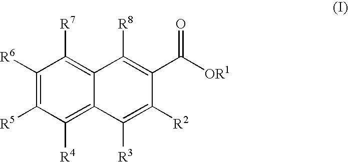

These substituted 2-naphthoic acids are effective as antagonists of the biological activity of the GPR105 protein. They are therefore useful for the treatment, control or prevention of disorders responsive to antagonism of this receptor, such as diabetes, in particular, Type 2 diabetes, hyperglycemia, insulin resistance, lipid disorders, obesity, atherosclerosis, and other conditions associated with the Metabolic Syndrome.

The present invention also relates to pharmaceutical compositions comprising the compounds of the present invention and a pharmaceutically acceptable carrier.

The present invention also relates to methods for the treatment, control, or prevention of disorders, diseases, or conditions responsive to antagonism of the GPR105 protein in a subject in need thereof by administering the compounds and pharmaceutical compositions of the present invention.

The present invention also relates to methods for the treatment, control, or prevention of diabetes, in particular, Type 2 diabetes, insulin resistance, obesity, lipid disorders, atherosclerosis, and other conditions associated with the Metabolic Syndrome by administering the compounds and pharmaceutical compositions of the present invention.

The present invention also relates to methods for the treatment, control, or prevention of obesity by administering the compounds of the present invention in combination with a therapeutically effective amount of one or more agents known to be useful to treat the condition.

The present invention also relates to methods for the treatment, control, or prevention of Type 2 diabetes by administering the compounds of the present invention in combination with a therapeutically effective amount of one or more agents known to be useful to treat the condition.

The present invention also relates to methods for the treatment, control, or prevention of atherosclerosis by administering the compounds of the present invention in combination with a therapeutically effective amount of one or more agents known to be useful to treat the condition.

The present invention also relates to methods for the treatment, control, or prevention of lipid disorders by administering the compounds of the present invention in combination with a therapeutically effective amount of one or more agents known to be useful to treat the condition.

The present invention also relates to methods for treating conditions associated with the Metabolic Syndrome by administering the compounds of the present invention in combination with a therapeutically effective amount of one or more agents known to be useful to treat such conditions.

### DETAILED DESCRIPTION OF THE INVENTION

The present invention relates to compounds of structural formula I:

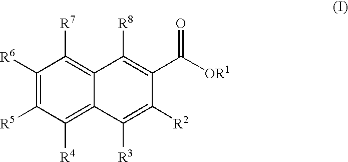

and pharmaceutically acceptable salts thereof, wherein
R¹ is selected from the group consisting of hydrogen,
   C₃₋₆ cycloalkyl,
   benzyl, and
   C₁₋₆ alkyl wherein alkyl is optionally substituted with hydroxy, amino, C₁₋₄ alkylamino, di-(C₁₋₄ alkyl)amino, aminocarbonyl, C₁₋₄ alkylaminocarbonyl, di-(C₁₋₄ alkyl)aminocarbonyl, C₁₋₄ alkylcarbonyloxy, C₁₋₄ alkyloxy, or one to five fluorines;
R² is hydrogen, fluorine, or hydroxy;
R³ is selected from the group consisting of:
   —(CH₂)ₘaryl,
   —(CH₂)ₘheteroaryl,
   —OCH₂-aryl,
   —OCH₂-heteroaryl,
   —(S)ᵣCH₂-aryl,
   —(S)ᵣCH₂-heteroaryl,
   —CH₂O-aryl,
   —CH₂O-heteroaryl,
   —CH₂(S)ᵣ-aryl, and
   —CH₂(S)ᵣ-heteroaryl;
      wherein any methylene (CH₂) carbon atom in R³ is optionally substituted with one to two groups independently selected from fluorine, hydroxy, and C₁₋₄ alkyl optionally substituted with one to three fluorines; or two substituents when on the same methylene (CH₂) group are taken together with the carbon atom to which they are attached to form a cyclopropyl group; and wherein aryl and heteroaryl are optionally substituted with one to three R^{c} substituents independently selected from the group consisting of:
   halogen,
   cyano,
   nitro,
   C₁₋₆ alkoxy, wherein alkoxy is optionally substituted with one to five substituents independently selected from fluorine, hydroxy, and C₁₋₃ alkoxy,
   C₁₋₆ alkyl, wherein alkyl is optionally substituted with one to five substituents independently selected from fluorine, hydroxy, and C₁₋₃ alkoxy,
   C₂₋₆ alkenyl, wherein alkenyl is optionally substituted with one to five substituents independently selected from fluorine, hydroxy, and C₁₋₃ alkoxy,
   (CH₂)ₙ-aryl,
   (CH₂)ₙ-heteroaryl,
   (CH₂)ₙ-heterocyclyl,
   (CH₂)ₙ—C₃₋₆ cycloalkyl,
   (CH₂)ₙ—OR⁹,
   (CH₂)ₙ—CO₂R⁹,
   (CH₂)ₙ—N(R⁹)₂,
   (CH₂)ₙ—CON(R⁹)₂,
   (CH₂)ₙ—OCON(R⁹)₂,
   (CH₂)ₙ—SO₂N(R⁹)₂,
   (CH₂)ₙ—SO₂N(R⁹)C(O)R⁹,
   (CH₂)ₙ—C(O)N(R⁹)SO₂R¹⁰,
   (CH₂)ₙ—S(O)ᵣR¹⁰,
   (CH₂)ₙ—NR¹¹SO₂R¹⁰;
   (CH₂)ₙ—NR¹¹CON(R⁹)₂,
   (CH₂)ₙ—NR¹¹COR⁹, and
   (CH₂)ₙ—NR¹¹CO₂R¹⁰;
      wherein aryl, heteroaryl, cycloalkyl, and heterocyclyl are optionally substituted with one to three substituents independently selected from halogen, hydroxy, C₁₋₄ alkyl, trifluoromethyl, and C₁₋₄ alkoxy; and wherein any methylene (CH₂) carbon atom in R^{c} is optionally substituted with one to two groups independently selected from fluorine, hydroxy, and C₁₋₄ alkyl optionally substituted with one to three fluorines; or two substituents when on the same methylene (CH₂) group are taken together with the carbon atom to which they are attached to form a cyclopropyl group;
R⁴, R⁵, R⁷, and R⁸ are each independently selected from the group consisting of:
   hydrogen,
   halogen,
   C₁₋₄ alkyl, optionally substituted with one to five fluorines,
   C₁₋₄ alkoxy, optionally substituted with one to five fluorines, and
   C₁₋₄ alkylthio, optionally substituted with one to five fluorines;
R⁶ is selected from the group consisting of:
   —(CH₂)ₘ-aryl,
   —(CH₂)ₘ-heteroaryl,
   —OCH₂-aryl,
   —OCH₂-heteroaryl,
   —(S)ᵣCH₂-aryl,
   —(S)ᵣCH₂-heteroaryl,
   —CH₂O-aryl,
   —CH₂O-heteroaryl,
   —CH₂(S)ᵣ-aryl, and
   —CH₂(S)ᵣ-heteroaryl;
      wherein any methylene (CH₂) carbon atom in R⁶ is optionally substituted with one to two groups independently selected from fluorine, hydroxy, and C₁₋₄ alkyl optionally substituted with one to three fluorines; or two substituents when on the same methylene (CH₂) group are taken together with the carbon atom to which they are attached to form a cyclopropyl group and wherein aryl and heteroaryl are optionally substituted with one to three R^{d} substituents independently selected from the group consisting of:
   halogen,
   cyano,
   C₁₋₄ alkyl, optionally substituted with one to five fluorines,
   C₁₋₄ alkoxy, optionally substituted with one to five fluorines,
   C₁₋₄ alkylthio, optionally substituted with one to five fluorines, and
   C₁₋₄ alkylsulfonyl, optionally substituted with one to five fluorines;
      each R⁹ is independently selected from the group consisting of hydrogen,
   C₁₋₆ alkyl,
   (CH₂)ₘ-aryl,
   (CH₂)ₘ-heteroaryl, and
   (CH₂)ₘC₃₋₆ cycloalkyl;
      wherein any individual methylene (CH₂) carbon atom in (CH₂)ₘ is optionally substituted with one to two substituents independently selected from fluorine, hydroxy, C₁₋₄ alkyl, and C₁₋₄ alkoxy, wherein alkyl and alkoxy are optionally substituted with one to five fluorines; or two substituents when on the same methylene (CH₂) group are taken together with the carbon atom to which they are attached to form a cyclopropyl group; and wherein alkyl, aryl, heteroaryl, and cycloalkyl are optionally substituted with one to three substituents independently selected from the group consisting of halogen, C₁₋₄ alkyl, and C₁₋₄ alkoxy; or two R⁹ groups substituents together with the nitrogen atom to which they are attached form a heterocyclic ring selected from azetidine, pyrrolidine, piperidine, piperazine, and morpholine wherein said heterocyclic ring is optionally substituted with one to three substituents independently selected from the group consisting of halogen, hydroxy, C₁₋₆ alkyl, and C₁₋₆ alkoxy, wherein alkyl and alkoxy are optionally substituted with one to five fluorines;
      each R¹⁰ is independently C₁₋₆ alkyl, wherein alkyl is optionally substituted with one to five substituents independently selected from fluorine and hydroxy;
      R¹¹ is hydrogen or R¹⁰;
      each n is independently an integer from 0 to 3;
      each m is independently an integer from 0 to 2; and
      each r is an integer from 0 to 2.

In one embodiment of the compounds of the present invention, R³ and R⁶ are each independently aryl or heteroaryl wherein R³ is optionally substituted with one to three R^{c} substituents as defined above, and R⁶ is optionally substituted with one to three R^{d} substituents as defined above. In a class of this embodiment, R³ is phenyl or thienyl each of which is optionally substituted with one to three R^{c} substituents as defined above. In a subclass of this class, R³ is 3-thienyl optionally substituted with one to two R^{c} substituents as defined above. In a second class of this embodiment, R⁶ is phenyl or pyridyl each of which is optionally substituted with one to three R^{c} substituents as defined above.

In a second embodiment of the compounds of the present invention, R³ is aryl or heteroaryl wherein R³ is optionally substituted with one to three R^{c} substituents as defined above; and R⁶ is —OCH₂-aryl or —OCH₂-heteroaryl wherein aryl and heteroaryl are optionally substituted with one to three R^{d} substituents as defined above. In a class of this embodiment, R³ is phenyl or thienyl wherein R³ is optionally substituted with one to three R^{c} substituents as defined above; and R⁶ is —OCH₂-phenyl or —OCH₂-pyridyl wherein phenyl and pyridyl are optionally substituted with one to three R^{d} substituents as defined above. In a subclass of this class, R³ is 3-thienyl optionally substituted with one to two R^{c} substituents as defined above.

In a third embodiment of the compounds of the present invention, R⁶ is aryl or heteroaryl wherein R⁶ is optionally substituted with one to three R^{d} substituents as defined above; and R³ is —OCH₂-aryl or —OCH₂-heteroaryl wherein aryl and heteroaryl are optionally substituted with one to three R^{c} substituents as defined above. In a class of this embodiment, R⁶ is phenyl optionally substituted with one to three R^{c} substituents as defined above; and R³ is —OCH₂-phenyl or —OCH₂-pyridyl wherein phenyl and pyridyl are optionally substituted with one to three R^{d} substituents as defined above.

In a fourth embodiment of the compounds of the present invention, R³ is —OCH₂-aryl or —OCH₂-heteroaryl wherein aryl and heteroaryl are optionally substituted with one to three R^{c} substituents as defined above; and R⁶ is —OCH₂-aryl or —OCH₂-heteroaryl wherein aryl and heteroaryl are optionally substituted with one to three R^{d} substituents as defined above. In a class of this embodiment, R³ is —OCH₂-phenyl or —OCH₂-pyridyl wherein phenyl and pyridyl are optionally substituted with one to three R^{c} substituents as defined above; and R⁶ is —OCH₂-phenyl wherein phenyl is optionally substituted with one to three R^{d} substituents as defined above.

In a fifth embodiment of the compounds of the present invention, R² is fluoro or hydrogen. In a class of this embodiment, R⁴, R⁵, R⁷, and R⁸ are each hydrogen.

In a sixth embodiment, R¹ is hydrogen. In a subclass of this embodiment, R² is fluoro or hydrogen, and R⁴, R⁵, R⁷, and R⁸ are each hydrogen.

In a seventh embodiment, R^{d} is selected from the group consisting of:
halogen,
C₁₋₃ alkyl, optionally substituted with one to three fluorines,
C₁₋₃ alkoxy, optionally substituted with one to three fluorines, and
C₁₋₃ alkylthio, optionally substituted with one to three fluorines.

In an eighth embodiment of the compounds of the present invention, R^{c} is selected from the group consisting of:
C₁₋₃ alkoxy, optionally substituted with one to three fluorines,
—CO₂R⁹,
—S(O)ᵣR¹⁰,
—C(O)R⁹,

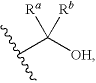

heterocyclyl, and
heteroaryl;
   wherein R⁹ and R¹⁰ are as defined above, and R^{a} and R^{b} are each independently hydrogen or methyl, wherein methyl is optionally substituted with one to three fluorines.

In a class of this embodiment, R⁹ is hydrogen or C₁₋₃ alkyl optionally substituted with one to three fluorines.

In a second class of this embodiment, R^{c} is selected from the group consisting of:

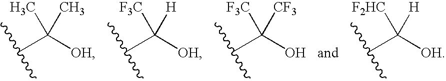

In a subclass of this class, R^{c} is selected from the group consisting of:

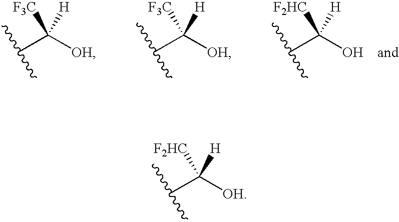

In a third class of this embodiment, R^{c} is heteroaryl or heterocyclyl each of which is optionally substituted with one to two substituents independently selected from halogen, hydroxy, C₁₋₄ alkyl, trifluoromethyl, and C₁₋₄ alkoxy. In a subclass of this class, R^{c} is piperidinyl, tetrazole or triazole each of which is optionally monosubstituted with halogen, hydroxy, C₁₋₄ alkyl, trifluoromethyl, or C₁₋₄ alkoxy.

In a ninth embodiment of the compounds of the present invention, R³ is phenyl monosubstituted at the para position with an R^{c} substituent as defined above.

In a tenth embodiment of the compounds of the present invention, R³ is

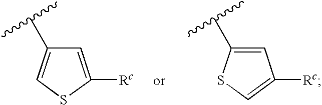

wherein the thienyl group is monosubstituted with R^{c} as defined above.

In an eleventh embodiment of the compounds of the present invention, R⁶ is phenyl monosubstituted at the para position with an R^{d} substituent as defined above.

In a twelfth embodiment of the compounds of the present invention, R⁶ is —OCH₂-phenyl wherein phenyl is substituted at the 2 and 6 positions each with an independent R^{d} substituent as defined above or phenyl is substituted at the 2, 4, and 6 positions each with an independent R^{d} substituent as defined above.

As used herein the following definitions are applicable.

“Alkyl”, as well as other groups having the prefix “alk”, such as alkoxy and alkanoyl, means carbon chains which may be linear or branched, and combinations thereof, unless the carbon chain is defined otherwise. Examples of alkyl groups include methyl, ethyl, propyl, isopropyl, butyl, sec- and tert-butyl, pentyl, hexyl, heptyl, octyl, nonyl, and the like.

“Cycloalkyl” means a saturated hydrocarbon containing one or more rings having a specified number of carbon atoms; the monocycle having the general formula CₙH₂ₙ, n being an integer corresponding to the number of carbon atoms in the ring. Examples of cycloalkyl include cyclopropyl, cyclobutyl, cyclopentyl, cyclohexyl, cycloheptyl, cyclooctyl, and the like. A cycloalkyl group generally is monocyclic unless stated otherwise. Cycloalkyl groups are saturated unless otherwise defined.

The term “alkenyl” refers to hydrocarbons of the specified number of carbon atoms having a carbon-carbon double bond anywhere in the chain. Examples of alkenyl groups include ethenyl, 1-propenyl, 1-butenyl, 2-butenyl, etc.

The term “alkoxy” refers to straight or branched chain alkoxides of the number of carbon atoms specified (e.g., C₁₋₆ alkoxy), or any number within this range [i.e., methoxy (MeO—), ethoxy, isopropoxy, etc.].

The term “alkylthio” refers to straight or branched chain alkylsulfides of the number of carbon atoms specified (e.g., C₁₋₆ alkylthio), or any number within this range [i.e., methylthio (MeS—), ethylthio, isopropylthio, etc.].

The term “alkylamino” refers to straight or branched alkylamines of the number of carbon atoms specified (e.g., C₁₋₆ alkylamino), or any number within this range [i.e., methylamino, ethylamino, isopropylamino, t-butylamino, etc.].

The term “alkylsulfonyl” refers to straight or branched chain alkylsulfones of the number of carbon atoms specified (e.g., C₁₋₆ alkylsulfonyl), or any number within this range [i.e., methylsulfonyl (MeSO₂—), ethylsulfonyl, isopropylsulfonyl, etc.].

The term “alkylsulfinyl” refers to straight or branched chain alkylsulfoxides of the number of carbon atoms specified (e.g., C₁₋₆ alkylsulfinyl), or any number within this range [i.e., methylsulfinyl (MeSO—), ethylsulfinyl, isopropylsulfinyl, etc.].

The term “alkyloxycarbonyl” refers to straight or branched chain esters of a carboxylic acid derivative of the present invention of the number of carbon atoms specified (e.g., C₁₋₆ alkyloxycarbonyl), or any number within this range [i.e., methyloxycarbonyl (MeOCO—), ethyloxycarbonyl, or butyloxycarbonyl].

“Aryl” means a mono- or polycyclic aromatic ring system containing carbon ring atoms. The preferred aryls are monocyclic or bicyclic 6-10 membered aromatic ring systems. Phenyl and naphthyl are preferred aryls. The most preferred aryl is phenyl.

“Heterocyclyl” refer to saturated or unsaturated non-aromatic rings or ring systems containing at least one heteroatom selected from O, S and N, further including the oxidized forms of sulfur, namely SO and SO₂. Examples of heterocycles include tetrahydrofuran (THF), dihydrofuran, 1,4-dioxane, oxacyclobutane (oxetane), thiacyclobutane (thietane), azacyclobutane (azetidine), morpholine, 1,4-dithiane, piperazine, piperidine, 1,3-dioxolane, imidazolidine, imidazoline, pyrroline, pyrrolidine, tetrahydropyran, dihydropyran, oxathiolane, dithiolane, 1,3-dioxane, 1,3-dithiane, oxathiane, thiomorpholine, 2-oxopiperidin-1-yl, 2-oxopyrrolidin-1-yl, 2-oxoazetidin-1-yl, 1,2,4-oxadiazin-5(6H)-one-3-yl, and the like.

“Heteroaryl” means an aromatic or partially aromatic heterocycle that contains at least one ring heteroatom selected from O, S and N. Heteroaryls thus includes heteroaryls fused to other kinds of rings, such as aryls, cycloalkyls and heterocycles that are not aromatic. Examples of heteroaryl groups include: pyrrolyl, isoxazolyl, isothiazolyl, pyrazolyl, pyridinyl, N-oxo-pyridinyl, oxazolyl, oxadiazolyl (in particular, 1,3,4-oxadiazol-2-yl and 1,2,4-oxadiazol-3-yl), thiadiazolyl, thiazolyl, imidazolyl, triazolyl, tetrazolyl, furyl, triazinyl, thienyl, pyrimidinyl, benzisoxazolyl, benzoxazolyl, benzothiazolyl, benzothiadiazolyl, dihydrobenzofuranyl, indolinyl, pyridazinyl, indazolyl, isoindolyl, dihydrobenzothienyl, indolizinyl, cinnolinyl, phthalazinyl, quinazolinyl, naphthyridinyl, carbazolyl, benzodioxolyl, quinoxalinyl, purinyl, furazanyl, isobenzylfuranyl, benzimidazolyl, benzofuranyl, benzothienyl, quinolyl, indolyl, isoquinolyl, dibenzofuranyl, 1,3-benzodioxolyl, imidazo[1,2-a]pyridinyl, imidazo[1,2-a]pyrimidinyl, [1,2,4-triazolo][4,3-a]pyridinyl, pyrazolo[1,5-a]pyridinyl, [1,2,4-triazolo][1,5-a]pyridinyl, 2-oxo-1,3-benzoxazolyl, 4-oxo-3H-quinazolinyl, 3-oxo-[1,2,4]-triazolo[4,3-a]-2H-pyridinyl, 5-oxo-[1,2,4]-4H-oxadiazolyl, 2-oxo-[1,3,4]-3H-oxadiazolyl, 2-oxo-1,3-dihydro-2H-imidazolyl, 3-oxo-2,4-dihydro-3H-1,2,4-triazolyl, 2,1,3-benzoxadiazolyl, and the like. For heterocyclyl and heteroaryl groups, rings and ring systems containing from 3-15 atoms are included, forming 1-3 rings. The atom of attachment of such heteroaryl group is either a carbon atom or a nitrogen where allowable by the rules of valency, such as pyrazol-1-yl and imidazol-1-yl.

“Halogen” refers to fluorine, chlorine, bromine and iodine.

### Optical Isomers—Diastereomers—Geometric Isomers—Tautomers:

Compounds of structural formula I may contain one or more asymmetric centers and can thus occur as racemates and racemic mixtures, single enantiomers, diastereomeric mixtures and individual diastereomers. The present invention is meant to comprehend all such isomeric forms of the compounds of structural formula I.

Compounds of structural formula I may be separated into their individual diastereoisomers by, for example, fractional crystallization from a suitable solvent, for example methanol or ethyl acetate or a mixture thereof, or via chiral chromatography using an optically active stationary phase. Absolute stereochemistry may be determined by X-ray crystallography of crystalline products or crystalline intermediates which are derivatized, if necessary, with a reagent containing an asymmetric center of known absolute configuration.

Alternatively, any stereoisomer of a compound of the general structural formula I may be obtained by stereospecific synthesis using optically pure starting materials or reagents of known absolute configuration.

If desired, racemic mixtures of the compounds may be separated so that the individual enantiomers are isolated. The separation can be carried out by methods well known in the art, such as the coupling of a racemic mixture of compounds to an enantiomerically pure compound to form a diastereomeric mixture, followed by separation of the individual diastereomers by standard methods, such as fractional crystallization or chromatography. The coupling reaction is often the formation of salts using an enantiomerically pure acid or base. The diasteromeric derivatives may then be converted to the pure enantiomers by cleavage of the added chiral residue. The racemic mixture of the compounds can also be separated directly by chromatographic methods utilizing chiral stationary phases, which methods are well known in the art.

Some of the compounds described herein contain olefinic double bonds, and unless specified otherwise, are meant to include both E and Z geometric isomers.

Some of the compounds described herein may exist as tautomers which have different points of attachment of hydrogen accompanied by one or more double bond shifts. For example, a ketone and its enol form are keto-enol tautomers. The individual tautomers as well as mixtures thereof are encompassed with compounds of the present invention.

### Salts:

It will be understood that, as used herein, references to the compounds of the present invention are meant to also include the pharmaceutically acceptable salts, and also salts that are not pharmaceutically acceptable when they are used as precursors to the free compounds or their pharmaceutically acceptable salts or in other synthetic manipulations.

The compounds of the present invention may be administered in the form of a pharmaceutically acceptable salt. The term “pharmaceutically acceptable salt” refers to salts prepared from pharmaceutically acceptable non-toxic bases or acids including inorganic or organic bases and inorganic or organic acids. Salts of basic compounds encompassed within the term “pharmaceutically acceptable salt” refer to non-toxic salts of the compounds of this invention which are generally prepared by reacting the free base with a suitable organic or inorganic acid. Representative salts of basic compounds of the present invention include, but are not limited to, the following: acetate, benzenesulfonate, benzoate, bicarbonate, bisulfate, bitartrate, borate, bromide, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, oleate, oxalate, pamoate (embonate), palmitate, pantothenate, phosphate/diphosphate, polygalacturonate, salicylate, stearate, sulfate, subacetate, succinate, tannate, tartrate, teoclate, tosylate, triethiodide and valerate. Furthermore, where the compounds of the invention carry an acidic moiety, suitable pharmaceutically acceptable salts thereof include, but are not limited to, salts derived from inorganic bases including aluminum, ammonium, calcium, copper, ferric, ferrous, lithium, magnesium, manganic, manganous, potassium, sodium, zinc, and the like. Particularly preferred are the ammonium, calcium, magnesium, potassium, and sodium salts. Salts derived from pharmaceutically acceptable organic non-toxic bases include salts of primary, secondary, and tertiary amines, cyclic amines, and basic ion-exchange resins, such as arginine, betaine, caffeine, choline, N,N-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like.

Also, in the case of a carboxylic acid (—COOH) or alcohol group being present in the compounds of the present invention, pharmaceutically acceptable optionally substituted lower alkyl esters of carboxylic acid derivatives, such as methyl, ethyl, dimethylamino-carbonylmethyl, or pivaloyloxymethyl, or acyl derivatives of alcohols, such as O-acetyl, O-pivaloyl, O-benzoyl, and β-aminoacyl, can be employed. Included are those esters and acyl groups known in the art for modifying the solubility or hydrolysis characteristics for use as sustained-release or prodrug formulations.

Solvated forms, in particular, hydrated forms, of the compounds of the present invention are included in the present invention as well.

### Administration and Dose Ranges

Any suitable route of administration may be employed for providing a mammal, especially a human, with an effective dose of a compound of the present invention. For example, oral, rectal, topical, parenteral, ocular, pulmonary, nasal, and the like may be employed. Dosage forms include tablets, troches, dispersions, suspensions, solutions, capsules, creams, ointments, aerosols, and the like. Preferably compounds of the present invention are administered orally.

In the treatment or prevention of conditions which require antagonism of GPR105 receptor activity, an appropriate dosage level will generally be about 0.01 to 500 mg per kg patient body weight per day which can be administered in single or multiple doses. Preferably, the dosage level will be about 0.1 to about 250 mg/kg per day; more preferably about 0.5 to about 100 mg/kg per day. A suitable dosage level may be about 0.01 to 250 mg/kg per day, about 0.05 to 100 mg/kg per day, or about 0.1 to 50 mg/kg per day. Within this range the dosage may be 0.05 to 0.5, 0.5 to 5 or 5 to 50 mg/kg per day. For oral administration, the compositions are preferably provided in the form of tablets containing 1.0 to 1000 mg of the active ingredient, particularly 1.0, 5.0, 10.0, 15.0. 20.0, 25.0, 50.0, 75.0, 100.0, 150.0, 200.0, 250.0, 300.0, 400.0, 500.0, 600.0, 750.0, 800.0, 900.0, and 1000.0 mg of the active ingredient for the symptomatic adjustment of the dosage to the patient to be treated. The compounds may be administered on a regimen of 1 to 4 times per day, preferably once or twice per day.

When treating or preventing Type 2 diabetes, hyperglycemia, hypertriglyceridemia, obesity or other diseases for which compounds of the present invention are indicated, generally satisfactory results are obtained when the compounds of the present invention are administered at a daily dosage of from about 0.1 mg to about 100 mg per kilogram of animal body weight, preferably given as a single daily dose or in divided doses two to six times a day, or in sustained release form. For most large mammals, the total daily dosage is from about 0.5 mg to about 1000 mg, preferably from about 1 mg to about 100 mg. In the case of a 70 kg adult human, the total daily dose will generally be from about 5 mg to about 350 mg. This dosage regimen may be adjusted to provide the optimal therapeutic response.

It will be understood, however, that the specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

### Pharmaceutical Compositions:

Another aspect of the present invention provides pharmaceutical compositions which comprises a compound of Formula I and a pharmaceutically acceptable carrier. The term “composition”, as in pharmaceutical composition, is intended to encompass a product comprising the active ingredient(s), and the inert ingredient(s) (pharmaceutically acceptable excipients) that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing a compound of Formula I, additional active ingredient(s), and pharmaceutically acceptable excipients.

Any suitable route of administration may be employed for providing a mammal, especially a human with an effective dosage of a compound of the present invention. For example, oral, sublingual, rectal, topical, parenteral, ocular, pulmonary, nasal, and the like may be employed. Dosage forms include tablets, troches, dispersions, suspensions, solutions, capsules, creams, ointments, aerosols, and the like.

The pharmaceutical compositions of the present invention comprise a compound of Formula I as an active ingredient or a pharmaceutically acceptable salt thereof, and may also contain a pharmaceutically acceptable carrier and optionally other therapeutic ingredients. The term “pharmaceutically acceptable salts” refers to salts prepared from pharmaceutically acceptable non-toxic bases or acids including inorganic bases or acids and organic bases or acids.

The compositions include compositions suitable for oral, sublingual, rectal, topical, parenteral (including subcutaneous, intramuscular, and intravenous), ocular (ophthalmic), pulmonary (aerosol inhalation), or nasal administration, although the most suitable route in any given case will depend on the nature and severity of the conditions being treated and on the nature of the active ingredient. They may be conveniently presented in unit dosage form and prepared by any of the methods well-known in the art of pharmacy.

For administration by inhalation, the compounds of the present invention are conveniently delivered in the form of an aerosol spray presentation from pressurized packs or nebulizers. The compounds may also be delivered as powders which may be formulated and the powder composition may be inhaled with the aid of an insufflation powder inhaler device. The preferred delivery systems for inhalation are metered dose inhalation (MDI) aerosol, which may be formulated as a suspension or solution of a compound of Formula I in suitable propellants, such as fluorocarbons or hydrocarbons and dry powder inhalation (DPI) aerosol, which may be formulated as a dry powder of a compound of Formula I with or without additional excipients.

Suitable topical formulations of a compound of formula I include transdermal devices, aerosols, creams, ointments, lotions, dusting powders, and the like.

In practical use, the compounds of Formula I can be combined as the active ingredient in intimate admixture with a pharmaceutical carrier according to conventional pharmaceutical compounding techniques. The carrier may take a wide variety of forms depending on the form of preparation desired for administration, e.g., oral or parenteral (including intravenous). In preparing the compositions for oral dosage form, any of the usual pharmaceutical media may be employed, such as, for example, water, glycols, oils, alcohols, flavoring agents, preservatives, coloring agents and the like in the case of oral liquid preparations, such as, for example, suspensions, elixirs and solutions; or carriers such as starches, sugars, microcrystalline cellulose, diluents, granulating agents, lubricants, binders, disintegrating agents and the like in the case of oral solid preparations such as, for example, powders, capsules and tablets, with the solid oral preparations being preferred over the liquid preparations. Because of their ease of administration, tablets and capsules represent the most advantageous oral dosage unit form in which case solid pharmaceutical carriers are obviously employed. If desired, tablets may be coated by standard aqueous or nonaqueous techniques.

In addition to the common dosage forms set out above, the compounds of Formula I may also be administered by controlled release means and/or delivery devices such as those described in U.S. Pat. Nos. 3,845,770; 3,916,899; 3,536,809; 3,598,123; 3,630,200 and 4,008,719.

Pharmaceutical compositions of the present invention suitable for oral administration may be presented as discrete units such as capsules, cachets or tablets each containing a predetermined amount of the active ingredient, as a powder or granules or as a solution or a suspension in an aqueous liquid, a non-aqueous liquid, an oil-in-water emulsion or a water-in-oil liquid emulsion. Such compositions may be prepared by any of the methods of pharmacy but all methods include the step of bringing into association the active ingredient with the carrier which constitutes one or more necessary ingredients. In general, the compositions are prepared by uniformly and intimately admixing the active ingredient with liquid carriers or finely divided solid carriers or both, and then, if necessary, shaping the product into the desired presentation. For example, a tablet may be prepared by compression or molding, optionally with one or more accessory ingredients. Compressed tablets may be prepared by compressing in a suitable machine, the active ingredient in a free-flowing form such as powder or granules, optionally mixed with a binder, lubricant, inert diluent, surface active or dispersing agent. Molded tablets may be made by molding in a suitable machine, a mixture of the powdered compound moistened with an inert liquid diluent. Desirably, each tablet contains from about 1 mg to about 500 mg of the active ingredient and each cachet or capsule contains from about 1 to about 500 mg of the active ingredient.

### Utilities and Combination Therapy:

The compounds of the present invention are useful for the control, prevention and treatment of conditions and diseases related to the Metabolic Syndrome; obesity; cardiovascular disease, such as atherosclerosis; diabetes, in particular, Type 2 diabetes; insulin resistance; cancer; neurological disease; and hepatic steatosis. The subject compounds are further useful in a method for the prevention or treatment of the aforementioned diseases, disorders and conditions in combination with other agents.

The compounds of the present invention may be used in combination with one or more other drugs in the treatment, prevention, suppression or amelioration of diseases or conditions for which compounds of Formula I or the other drugs may have utility, where the combination of the drugs together are safer or more effective than either drug alone. Such other drug(s) may be administered, by a route and in an amount commonly used therefor, contemporaneously or sequentially with a compound of Formula I. When a compound of Formula I is used contemporaneously with one or more other drugs, a pharmaceutical composition in unit dosage form containing such other drugs and the compound of Formula I is preferred. However, the combination therapy may also include therapies in which the compound of formula I and one or more other drugs are administered on different overlapping schedules. It is also contemplated that when used in combination with one or more other active ingredients, the compounds of the present invention and the other active ingredients may be used in lower doses than when each is used singly. Accordingly, the pharmaceutical compositions of the present invention include those that contain one or more other active ingredients, in addition to a compound of Formula I.

Examples of other active ingredients that may be administered in combination with a compound of the present invention, and either administered separately or in the same pharmaceutical composition, include, but are not limited to:

(a) other dipeptidyl peptidase IV (DPP-4) inhibitors;

(b) insulin sensitizers including (i) PPARγ agonists, such as the glitazones (e.g. troglitazone, pioglitazone, englitazone, MCC-555, rosiglitazone, balaglitazone, and the like) and other PPAR ligands, including PPARα/γ dual agonists, such as muraglitazar, naveglitazar, tesaglitazar, and TAK-559; PPARα agonists, such as fenofibric acid derivatives (gemfibrozil, clofibrate, fenofibrate and bezafibrate); and selective PPARγ modulators (SPPARγM's), such as disclosed in WO 02/060388, WO 02/08188, WO 2004/019869, WO 2004/020409, WO 2004/020408, and WO 2004/066963; (ii) biguanides such as metformin and phenformin, and (iii) protein tyrosine phosphatase-1B (PTP-1B) inhibitors;

(c) insulin or insulin mimetics;

(d) sulfonylureas and other insulin secretagogues, such as tolbutamide, glyburide, glipizide, glimepiride, and meglitinides, such as nateglinide and repaglinide;

(e) α-glucosidase inhibitors (such as acarbose and miglitol);

(f) glucagon receptor antagonists, such as those disclosed in WO 97/16442; WO 98/04528, WO 98/21957; WO 98/22108; WO 98/22109; WO 99/01423, WO 00/39088, and WO 00/69810; WO 2004/050039; and WO 2004/069158;

(g) GLP-1, GLP-1 analogues or mimetics, and GLP-1 receptor agonists, such as exendin-4 (exenatide), liraglutide (N,N-2211), CJC-1131, LY-307161, and those disclosed in WO 00/42026 and WO 00/59887;

(h) GIP and GIP mimetics, such as those disclosed in WO 00/58360, and GIP receptor agonists;

(i) PACAP, PACAP mimetics, and PACAP receptor agonists such as those disclosed in WO 01/23420;

(j) cholesterol lowering agents such as (i) HMG-CoA reductase inhibitors (lovastatin, simvastatin, pravastatin, cerivastatin, fluvastatin, atorvastatin, itavastatin, and rosuvastatin, and other statins), (ii) sequestrants (cholestyramine, colestipol, and dialkylaminoalkyl derivatives of a cross-linked dextran), (iii) nicotinyl alcohol, nicotinic acid or a salt thereof, (iv) PPARα agonists such as fenofibric acid derivatives (gemfibrozil, clofibrate, fenofibrate and bezafibrate), (v) PPARα/γ dual agonists, such as naveglitazar and muraglitazar, (vi) inhibitors of cholesterol absorption, such as beta-sitosterol and ezetimibe, (vii) acyl CoA:cholesterol acyltransferase inhibitors, such as avasimibe, and (viii) antioxidants, such as probucol;

(k) PPARδ agonists, such as those disclosed in WO 97/28149;

(l) antiobesity compounds, such as fenfluramine, dexfenfluramine, phentermine, sibutramine, orlistat, neuropeptide Y₁ or Y₅ antagonists, CB1 receptor inverse agonists and antagonists, β₃ adrenergic receptor agonists, melanocortin-receptor agonists, in particular melanocortin-4 receptor agonists, ghrelin antagonists, bombesin receptor agonists (such as bombesin receptor subtype-3 agonists), cholecystokinin 1 (CCK-1) receptor agonists, and melanin-concentrating hormone (MCH) receptor antagonists;

(m) ileal bile acid transporter inhibitors;

(n) agents intended for use in inflammatory conditions such as aspirin, non-steroidal anti-inflammatory drugs (NSAIDs), glucocorticoids, azulfidine, and selective cyclooxygenase-2 (COX-2) inhibitors;

(o) antihypertensive agents, such as ACE inhibitors (enalapril, lisinopril, captopril, quinapril, tandolapril), A-II receptor blockers (losartan, candesartan, irbesartan, valsartan, telmisartan, and eprosartan), beta blockers and calcium channel blockers;

(p) glucokinase activators (GKAs), such as those disclosed in WO 03/015774; WO 04/076420; and WO 04/081001;

(q) inhibitors of 11β-hydroxysteroid dehydrogenase type 1, such as those disclosed in U.S. Pat. No. 6,730,690; WO 03/104207; and WO 04/058741;

(r) inhibitors of cholesteryl ester transfer protein (CETP), such as torcetrapib; and

(s) inhibitors of fructose 1,6-bisphosphatase, such as those disclosed in U.S. Pat. Nos. 6,054,587; 6,110,903; 6,284,748; 6,399,782; and 6,489,476.

Dipeptidyl peptidase-IV inhibitors that can be combined with compounds of structural formula I include those disclosed in U.S. Pat. No. 6,699,871; WO 02/076450 (3 Oct. 2002); WO 03/004498 (16 Jan. 2003); WO 03/004496 (16 Jan. 2003); EP 1 258 476 (20 Nov. 2002); WO 02/083128 (24 Oct. 2002); WO 02/062764 (15 Aug. 2002); WO 03/000250 (3 Jan. 2003); WO 03/002530 (9 Jan. 2003); WO 03/002531 (9 Jan. 2003); WO 03/002553 (9 Jan. 2003); WO 03/002593 (9 Jan. 2003); WO 03/000180 (3 Jan. 2003); WO 03/082817 (9 Oct. 2003); WO 03/000181 (3 Jan. 2003); WO 04/007468 (22 Jan. 2004); WO 04/032836 (24 Apr. 2004); WO 04/037169 (6 May 2004); and WO 04/043940 (27 May 2004). Specific DPP-4 inhibitor compounds include isoleucine thiazolidide (P32/98); NVP-DPP-728; vildagliptin (LAF 237); P93/01; and saxagliptin (BMS 477118).

Antiobesity compounds that can be combined with compounds of structural formula I include fenfluramine, dexfenfluramine, phentermine, sibutramine, orlistat, neuropeptide Y₁ or Y₅ antagonists, cannabinoid CBI receptor antagonists or inverse agonists, melanocortin receptor agonists, in particular, melanocortin-4 receptor agonists, ghrelin antagonists, bombesin receptor agonists, and melanin-concentrating hormone (MCH) receptor antagonists. For a review of anti-obesity compounds that can be combined with compounds of structural formula I, see S. Chaki et al., “Recent advances in feeding suppressing agents: potential therapeutic strategy for the treatment of obesity,” Expert Opin. Ther. Patents, 11: 1677-1692 (2001); D. Spanswick and K. Lee, “Emerging antiobesity drugs,” Expert Opin. Emerging Drugs, 8: 217-237 (2003); and J. A. Fernandez-Lopez, et al., “Pharmacological Approaches for the Treatment of Obesity,” Drugs, 62: 915-944 (2002).

Neuropeptide Y5 antagonists that can be combined with compounds of structural formula I include those disclosed in U.S. Pat. No. 6,335,345 (1 Jan. 2002) and WO 01/14376 (1 Mar. 2001); and specific compounds identified as GW 59884A; GW 569180A; LY366377; and CGP-71683A.

Cannabinoid CB1 receptor antagonists that can be combined with compounds of formula I include those disclosed in PCT Publication WO 03/007887; U.S. Pat. No. 5,624,941, such as rimonabant; PCT Publication WO 02/076949, such as SLV-319; U.S. Pat. No. 6,028,084; PCT Publication WO 98/41519; PCT Publication WO 00/10968; PCT Publication WO 99/02499; U.S. Pat. No. 5,532,237; U.S. Pat. No. 5,292,736; PCT Publication WO 05/000809; PCT Publication WO 03/086288; PCT Publication WO 03/087037; PCT Publication WO 04/048317; PCT Publication WO 03/007887; PCT Publication WO 03/063781; PCT Publication WO 03/075660; PCT Publication WO 03/077847; PCT Publication WO 03/082190; PCT Publication WO 03/082191; PCT Publication WO 03/087037; PCT Publication WO 03/086288; PCT Publication WO 04/012671; PCT Publication WO 04/029204; PCT Publication WO 04/040040; PCT Publication WO 01/64632; PCT Publication WO 01/64633; and PCT Publication WO 01/64634.

Melanocortin-4 receptor (MC4R) agonists useful in the present invention include, but are not limited to, those disclosed in U.S. Pat. No. 6,294,534, U.S. Pat. Nos. 6,350,760, 6,376,509, 6,410,548, 6,458,790, U.S. Pat. No. 6,472,398, U.S. Pat. No. 5,837,521, U.S. Pat. No. 6,699,873, which are hereby incorporated by reference in their entirety; in US Patent Application Publication Nos. US 2002/0004512, US2002/0019523, US2002/0137664, US2003/0236262, US2003/0225060, US2003/0092732, US2003/109556, US 2002/0177151, US 2002/187932, US 2003/0113263, which are hereby incorporated by reference in their entirety; and in WO 99/64002, WO 00/74679, WO 02/15909, WO 01/70708, WO 01/70337, WO 01/91752, WO 02/068387, WO 02/068388, WO 02/067869, WO 03/007949, WO 2004/024720, WO 2004/089307, WO 2004/078716, WO 2004/078717, WO 2004/037797, WO 01/58891, WO 02/070511, WO 02/079146, WO 03/009847, WO 03/057671, WO 03/068738, WO 03/092690, WO 02/059095, WO 02/059107, WO 02/059108, WO 02/059117, WO 02/085925, WO 03/004480, WO 03/009850, WO 03/013571, WO 03/031410, WO 03/053927, WO 03/061660, WO 03/066597, WO 03/094918, WO 03/099818, WO 04/037797, WO 04/048345, WO 02/018327, WO 02/080896, WO 02/081443, WO 03/066587, WO 03/066597, WO 03/099818, WO 02/062766, WO 03/000663, WO 03/000666, WO 03/003977, WO 03/040107, WO 03/040117, WO 03/040118, WO 03/013509, WO 03/057671, WO 02/079753, WO 02/092566, WO 03/-093234, WO 03/095474, and WO 03/104761.

The potential utility of safe and effective activators of glucokinase (GKAs) for the treatment of diabetes is discussed in J. Grimsby et al., “Allosteric Activators of Glucokinase: Potential Role in Diabetes Therapy,” Science, 301: 370-373 (2003).

One particular aspect of combination therapy concerns a method of treating a condition selected from the group consisting of hypercholesterolemia, atherosclerosis, low HDL levels, high LDL levels, hyperlipidemia, hypertriglyceridemia, and dyslipidemia, in a mammalian patient in need of such treatment comprising administering to the patient a therapeutically effective amount of a compound of structural formula I and an HMG-CoA reductase inhibitor.

More particularly, this aspect of combination therapy concerns a method of treating a condition selected from the group consisting of hypercholesterolemia, atherosclerosis, low HDL levels, high LDL levels, hyperlipidemia, hypertriglyceridemia and dyslipidemia in a mammalian patient in need of such treatment wherein the HMG-CoA reductase inhibitor is a statin selected from the group consisting of lovastatin, simvastatin, pravastatin, cerivastatin, fluvastatin, atorvastatin, and rosuvastatin.

In another aspect of the invention, a method of reducing the risk of developing a condition selected from the group consisting of hypercholesterolemia, atherosclerosis, low HDL levels, high LDL levels, hyperlipidemia, hypertriglyceridemia and dyslipidemia, and the sequelae of such conditions is disclosed comprising administering to a mammalian patient in need of such treatment a therapeutically effective amount of a compound of structural formula I and an HMG-CoA reductase inhibitor.

In another aspect of the invention, a method for delaying the onset or reducing the risk of developing atherosclerosis in a human patient in need of such treatment is disclosed comprising administering to said patient an effective amount of a compound of structural formula I and an HMG-CoA reductase inhibitor.

More particularly, a method for delaying the onset or reducing the risk of developing atherosclerosis in a human patient in need of such treatment is disclosed, wherein the HMG-CoA reductase inhibitor is a statin selected from the group consisting of: lovastatin, simvastatin, pravastatin, cerivastatin, fluvastatin, atorvastatin, and rosuvastatin.

In another aspect of the invention, a method for delaying the onset or reducing the risk of developing atherosclerosis in a human patient in need of such treatment is disclosed, wherein the HMG-Co A reductase inhibitor is a statin and further comprising administering a cholesterol absorption inhibitor.

More particularly, in another aspect of the invention, a method for delaying the onset or reducing the risk of developing atherosclerosis in a human patient in need of such treatment is disclosed, wherein the HMG-Co A reductase inhibitor is a statin and the cholesterol absorption inhibitor is ezetimibe.

In another aspect of the invention, a pharmaceutical composition is disclosed which comprises:

(1) a compound of structural formula I;
(2) one or more compounds selected from the group consisting of:

(a) dipeptidyl peptidase-IV (DPP-4) inhibitors;

(b) insulin sensitizers including (i) PPARγ agonists, such as the glitazones (e.g. troglitazone, pioglitazone, englitazone, MCC-555, rosiglitazone, balaglitazone, and the like) and other PPAR ligands, including PPARα/γ dual agonists, such as KRP-297, muraglitazar, naveglitazar, Galida, TAK-559, PPARα agonists, such as fenofibric acid derivatives (gemfibrozil, clofibrate, fenofibrate and bezafibrate), and selective PPARγ modulators (SPPARγM's), such as disclosed in WO 02/060388, WO 02/08188, WO 2004/019869, WO 2004/020409, WO 2004/020408, and WO 2004/066963; (ii) biguanides such as metformin and phenformin, and (iii) protein tyrosine phosphatase-1B (PTP-1B) inhibitors;

(c) insulin or insulin mimetics;

(d) sulfonylureas and other insulin secretagogues, such as tolbutamide, glyburide, glipizide, glimepiride, and meglitinides, such as nateglinide and repaglinide;

(e) α-glucosidase inhibitors (such as acarbose and miglitol);

(f) glucagon receptor antagonists, such as those disclosed in WO 98/04528, WO 99/01423, WO 00/39088, and WO 00/69810;

(g) GLP-1, GLP-1 analogues or mimetics, and GLP-1 receptor agonists, such as exendin-4 (exenatide), liraglutide (N,N-2211), CJC-1131, LY-307161, and those disclosed in WO 00/42026 and WO 00/59887;

(h) GIP and GIP mimetics, such as those disclosed in WO 00/58360, and GIP receptor agonists;

(i) PACAP, PACAP mimetics, and PACAP receptor agonists such as those disclosed in WO 01/23420;

(j) cholesterol lowering agents such as (i) HMG-CoA reductase inhibitors (lovastatin, simvastatin, pravastatin, cerivastatin, fluvastatin, atorvastatin, itavastatin, and rosuvastatin, and other statins), (ii) sequestrants (cholestyramine, colestipol, and dialkylaminoalkyl derivatives of a cross-linked dextran), (iii) nicotinyl alcohol, nicotinic acid or a salt thereof, (iv) PPARα agonists such as fenofibric acid derivatives (gemfibrozil, clofibrate, fenofibrate and bezafibrate), (v) PPARα/γ dual agonists, such as naveglitazar and muraglitazar, (vi) inhibitors of cholesterol absorption, such as beta-sitosterol and ezetimibe, (vii) acyl CoA:cholesterol acyltransferase inhibitors, such as avasimibe, and (viii) antioxidants, such as probucol;

(k) PPARδ agonists, such as those disclosed in WO 97/28149;

(l) antiobesity compounds, such as fenfluramine, dexfenfluramine, phentermine, sibutramine, orlistat, neuropeptide Y1 or Y5 antagonists, CB1 receptor inverse agonists and antagonists, β3 adrenergic receptor agonists, melanocortin-receptor agonists, in particular melanocortin-4 receptor agonists, ghrelin antagonists, bombesin receptor agonists (such as bombesin receptor subtype-3 agonists), melanin-concentrating hormone (MCH) receptor antagonists, and inhibitors of microsomal triglyceride transfer protein;

(m) ileal bile acid transporter inhibitors;

(n) agents intended for use in inflammatory conditions such as aspirin, non-steroidal anti-inflammatory drugs (NSAIDs), glucocorticoids, azulfidine, and selective cyclooxygenase-2 (COX-2) inhibitors;

(o) antihypertensive agents, such as ACE inhibitors (enalapril, lisinopril, captopril, quinapril, tandolapril), A-II receptor blockers (losartan, candesartan, irbesartan, valsartan, telmisartan, and eprosartan), beta blockers and calcium channel blockers;

(p) glucokinase activators (GKAs), such as those disclosed in WO 03/015774; WO 04/076420; and WO 04/081001;

(q) inhibitors of 11β-hydroxysteroid dehydrogenase type 1, such as those disclosed in U.S. Pat. No. 6,730,690; WO 03/104207; and WO 04/058741;

(r) inhibitors of cholesteryl ester transfer protein (CETP), such as torcetrapib, and structures disclosed in WO 06/014413 and WO 06/014357;

(s) inhibitors of fructose 1,6-bisphosphatase, such as those disclosed in U.S. Pat. Nos. 6,054,587; 6,110,903; 6,284,748; 6,399,782; and 6,489,476;

(t) acetyl CoA carboxylase-1 and/or -2 inhibitors; and

(u) AMPK activators;

(v) SCD1 inhibitors; and

(w) inhibitors of sodium-glucose co-transporter (SGLT-2); and

(3) a pharmaceutically acceptable carrier.

When a compound of the present invention is used contemporaneously with one or more other drugs, a pharmaceutical composition containing such other drugs in addition to the compound of the present invention is preferred. Accordingly, the pharmaceutical compositions of the present invention include those that also contain one or more other active ingredients, in addition to a compound of the present invention.

The weight ratio of the compound of the present invention to the second active ingredient may be varied and will depend upon the effective dose of each ingredient. Generally, an effective dose of each will be used. Thus, for example, when a compound of the present invention is combined with another agent, the weight ratio of the compound of the present invention to the other agent will generally range from about 1000:1 to about 1:1000, preferably about 200:1 to about 1:200. Combinations of a compound of the present invention and other active ingredients will generally also be within the aforementioned range, but in each case, an effective dose of each active ingredient should be used.

In such combinations the compound of the present invention and other active agents may be administered separately or in conjunction. In addition, the administration of one element may be prior to, concurrent to, or subsequent to the administration of other agent(s).

### Assays For Determining Biological Activity:

### A. Cell-Binding Assay:

A stable HEK clonal cell line expressing the chimpanzee GPR105 protein and the chimeric G protein Gqi5 was developed. The chimeric Gqi5 forces the coupling of GPR105 through the Gq (calcium) pathway and allows for monitoring of calcium signaling using a calcium binding fluorescent dye and the FLIPR (fluorometric imaging plate reader, MDS Sciex). 12,500 HEK/GPR105/Gqi5 expressing cells were plated in 25 μL Dulbecco's Modified Eagle's Medium (DMEM) containing 10% fetal bovine serum (FBS) onto 384-well, poly-D-lysine coated plates. Cells were incubated overnight at 37° C. and 5% CO₂ to form a monolayer. On the following day, 30 μl, of fluorescent no-wash dye was added to the cell monolayer and the plate was incubated for 60 mM at 37° C., 5% CO₂. 250 mL of compound in 100% DMSO was added to cell/dye incubation using acoustic dispensing (Echo™, Labcyte). Following a 20 minute incubation of compound at room temperature, 6.25 μL of UDP-glucose agonist (at EC₈₀) in Hank's Balanced Salt Solution (HBSS) containing 20 mM Hepes was added to cells and Ca²⁺ signaling was monitored by FLIPR. Quantitation of the % inhibition of Ca²⁺ signaling by antagonist was calculated using the maximum fluorescent signal detected. IC₅₀'s for the compounds of structural formula (I) were calculated as follows:

a.) Basal=incubation of cells+DMSO+Buffer;
b.) EC₈₀=incubation of cells of DMSO+agonist to achieve 80% maximum stimulation of calcium release;
c.) Compound=incubation of cells+antagonist in DMSO+EC80 agonist;
d.) Calcium release monitored by Fluorescence (RFU relative fluorescence units) using the FLIPR;
e.) The percentage of inhibition was calculated according to the equation: (1−(compound sample−Basal)/(EC80−Basal))×100;
f.) The percentage of inhibition at each dose was plotted, the Four Parameter Logistic Fit performed to draw the curve and the IC₅₀ is the compound dose where the % inhibition=50%.

The compounds of structural formula I, particularly the compounds of Examples 1 through 37 below, exhibit an inhibition constant IC₅₀ of less than 1 micromolar (μM) and more typically less than 100 nanomolar (nM). Representative inhibition IC₅₀'s for compounds of the present invention against the chimpanzee GPR105 protein are provided in Table 1:

TABLE 1

| | IC₅₀ |
|---|---|
| Representative Compound | (nM) |
| 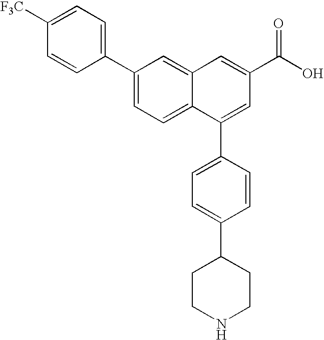 | 2.2 |
| 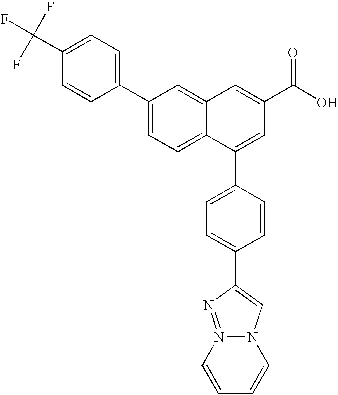 | 3.0 |
| 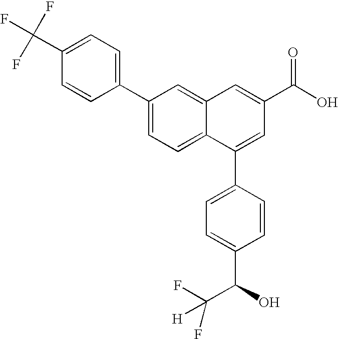 | 5.4 |
| 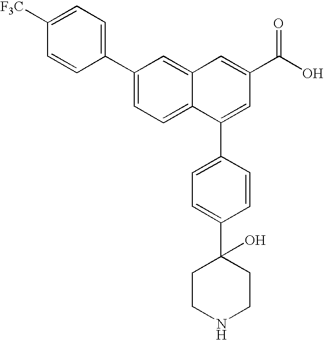 | 6.3 |
| 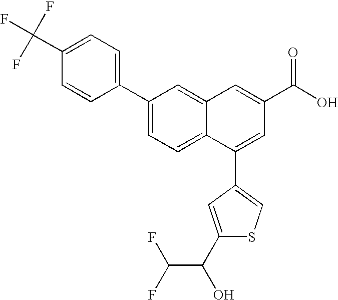 | 7.1 |
| 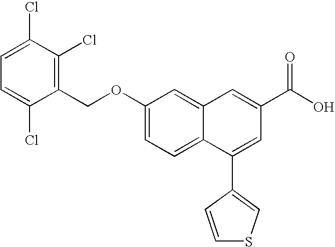 | 7.3 |
| 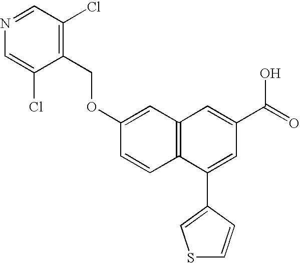 | 7.5 |
| 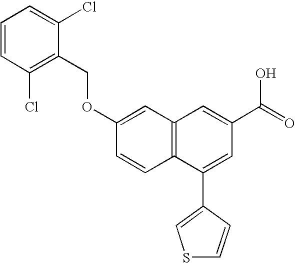 | 9.4 |
| 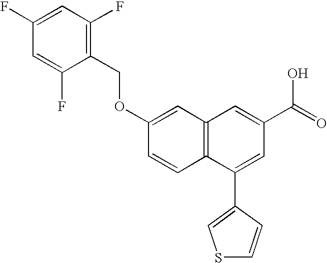 | 10.5 |
| 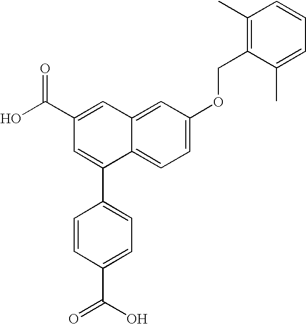 | 11.0 |
| 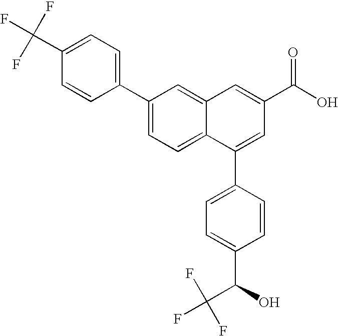 | 11.3 |
| 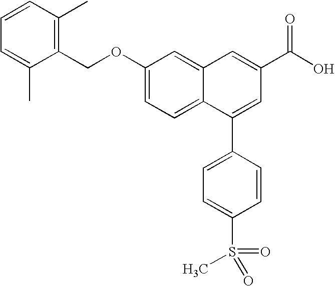 | 11.9 |
| 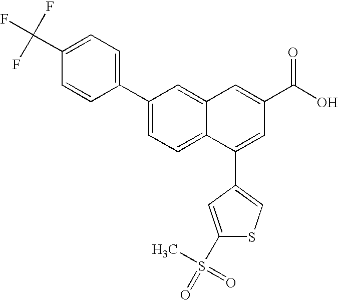 | 12.2 |
| 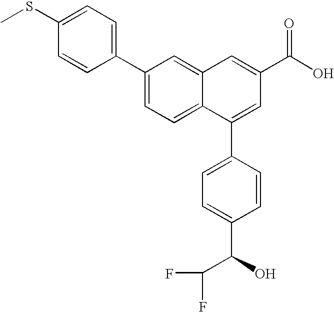 | 12.4 |
| 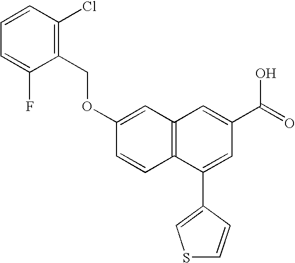 | 12.5 |
| 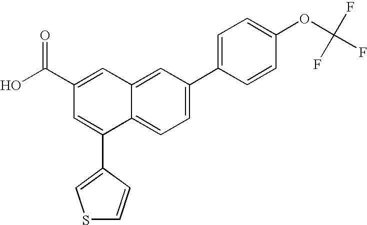 | 12.7 |
| 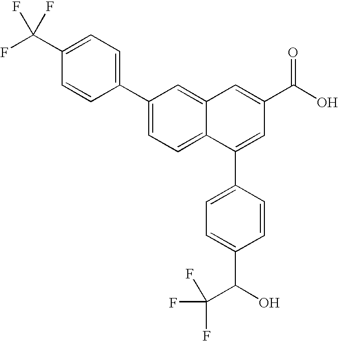 | 13.0 |
| 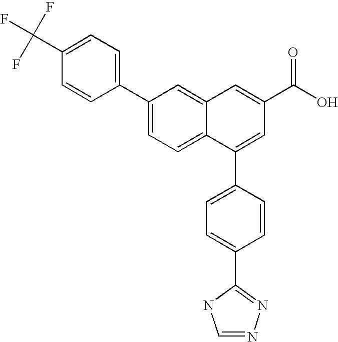 | 13.3 |
| 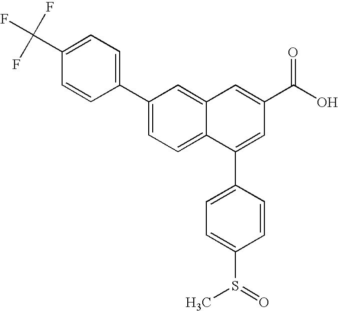 | 13.9 |
| 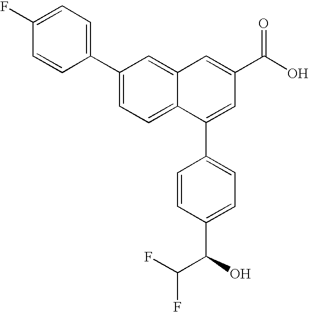 | 14.0 |
| 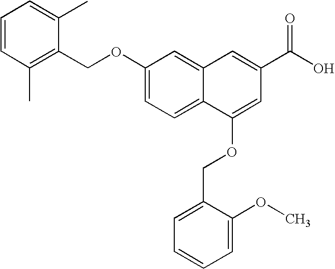 | 14.1 |
| 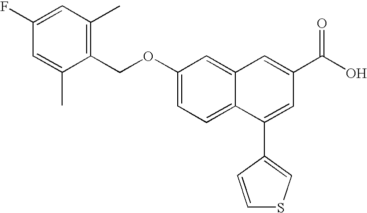 | 14.4 |
| 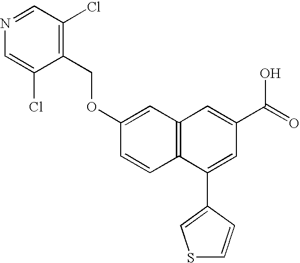 | 15.1 |
| 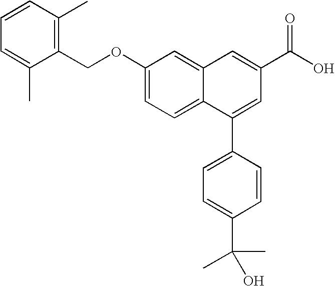 | 16.1 |
| 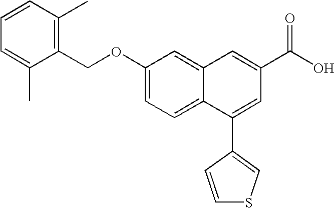 | 16.3 |
| 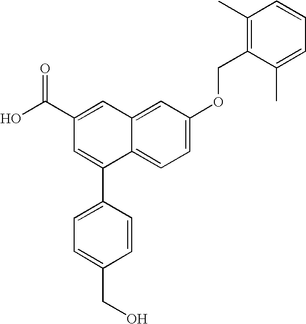 | 16.6 |
| 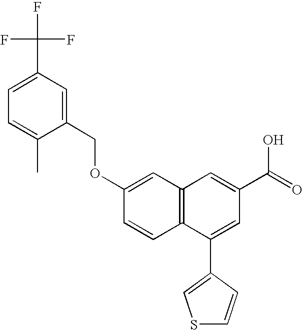 | 16.8 |
| 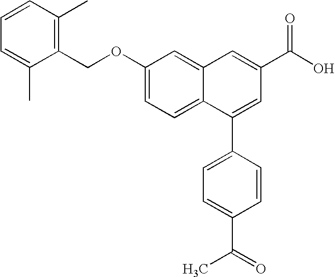 | 17.5 |
| 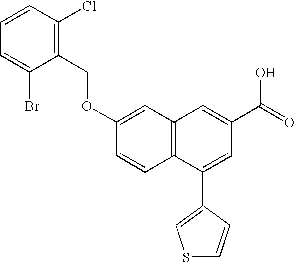 | 17.8 |
| 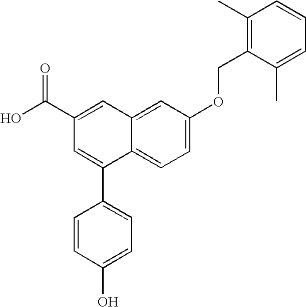 | 18.4 |
| 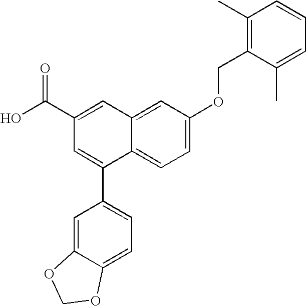 | 19.4 |
| 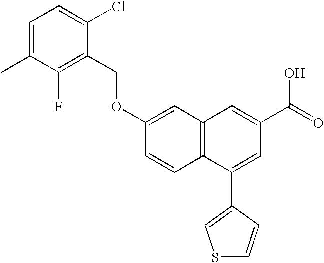 | 20.3 |
| 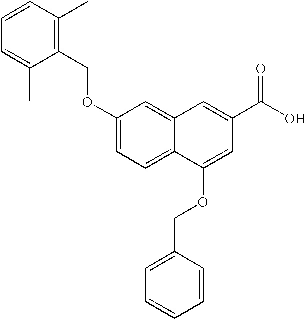 | 21.2 |
| 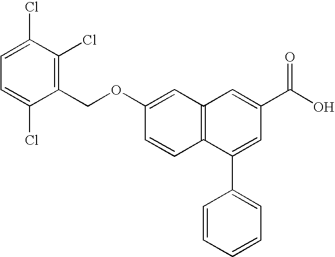 | 21.7 |
| 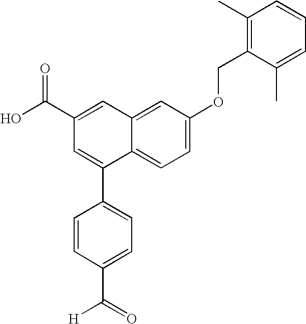 | 22.5 |
| 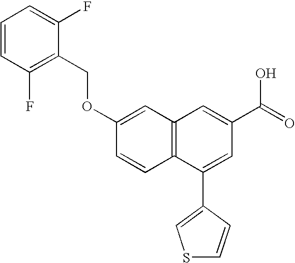 | 22.6 |
| 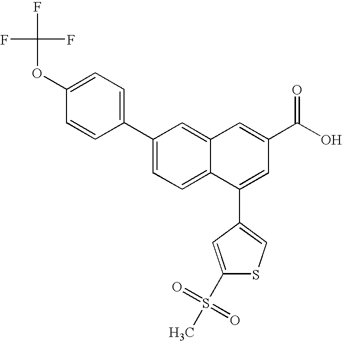 | 23.9 |
| 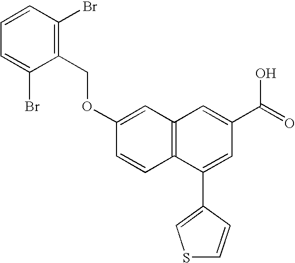 | 24.3 |
| 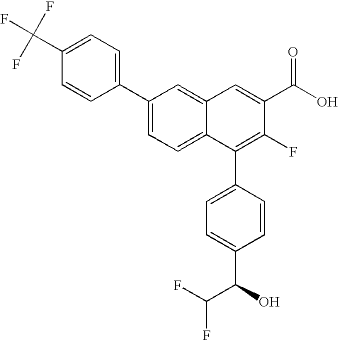 | 24.8 |
| 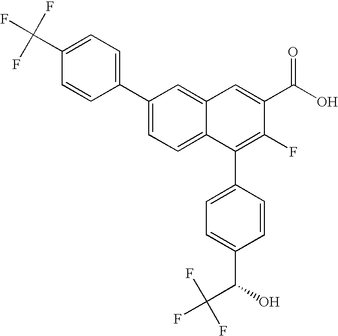 | 25.5 |
| 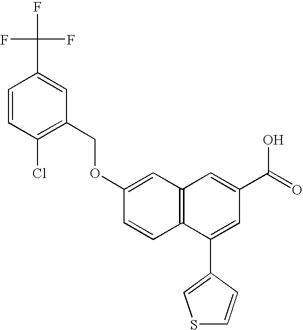 | 26.0 |

### B. Diet-Induced Obese [DIO] Mouse Protocol

a. Established DIO [eDIO]

C57B1/6 mice at 6 weeks of age are placed on a high fat diet [Research Diets D12492] consisting of fat, carbohydrate and protein at 60:20:20 kcal %. Mice of at least 20 weeks of age [14 weeks on the high fat diet] are used for the experiments. One week before compound treatment, the mice are dosed orally with the study vehicle to acclimate the mice with the dosing procedure [mock dosing]. A test compound or the vehicle is then administered orally either once or twice daily for a two-week period. Body weight, food consumption, and plasma compound levels from a satellite group of mice are measured at regular intervals during the study period. In this paradigm, loss of body weight from an established obesity state is the target endpoint. At the end of the study, additional endpoints such as plasma insulin, leptin, adiponectin levels, plasma glucose, blood lipid profile, blood cell counts and tissue compound levels are measured as needed.

b. Growing DIO [gDIO]

The protocol is similar to that used for eDIO mice except that mock dosing followed by compound treatment is given to young growing mice at 6-7 weeks of age at the same time when they are fed with the high fat diet. In this case, prevention of body weight gain is measured. Terminal endpoints as listed above are obtained as appropriate.

### Methods of Synthesis of the Compounds of Structural Formula (I):

The compounds of structural formula I can be prepared according to the procedures of the following Schemes and Examples, using appropriate materials and are further exemplified by the following specific examples. The compounds illustrated in the examples are not, however, to be construed as forming the only genus that is considered as the invention. The Examples further illustrate details for the preparation of the compounds of the present invention. Those skilled in the art will readily understand that known variations of protecting groups, as well as of the conditions and processes of the following preparative procedures, can be used to prepare these compounds. It is also understood that whenever a chemical reagent, such as a boronic acid or a boronate, is not commercially available, such a chemical reagent can be readily prepared following one of numerous methods described in the literature. All temperatures are degrees Celsius unless otherwise noted. Mass spectra (MS) were measured either by electrospray ion-mass spectroscopy (ESMS) or by atmospheric pressure chemical ionization mass spectroscopy (APCI). By “drying as usual” is meant drying an organic solution with either anhydrous sodium sulfate or anhydrous magnesium sulfate.

### LIST OF ABBREVIATIONS

Alk=alkyl
APCI=atmospheric pressure chemical ionization
Ar=aryl
Boc=tert-butoxycarbonyl
BOC₂O=Di-tert-butyl dicarbonate
br=broad
Cbz=benzyloxycarbonyl
CH₂Cl₂=dichloromethane
d=doublet
DCM=dichloromethane
DIPEA=N,N-diisopropylethylamine
DMAP=4-dimethylaminopyridine
DMF=N,N-dimethylformamide
DMSO=dimethylsulfoxide
EA=ethyl acetate
ESI=electrospray ionization
EtOAc=ethyl acetate
Et₃N=triethylamine
h=hour(s)
H=hexanes
HOAc=acetic acid
KOH=potassium hydroxide
LC-MS=liquid chromatography-mass spectroscopy
LiOH=lithium hydroxide
m=multiplet
min=minutes
MeOH=methyl alcohol
MgSO₄=magnesium sulfate
MS=mass spectroscopy
MTBE=methyl tert-butyl ether
NaOH=sodium hydroxide
Na₂SO₄=sodium sulfate
NH₄OAc=ammonium acetate
NH₄Cl=ammonium chloride
NMR=nuclear magnetic resonance spectroscopy
ON=overnight
PG=protecting group
RT or rt=room temperature
s=singlet
t=triplet
Tf₂O=triflic anhydride or trifluoromethanesulfonic anhydride
THF=tetrahydrofuran
TFA=trifluoroacetic acid
TLC=thin-layer chromatography
TsCl=p-toluenesulfonyl chloride

### Method A (Scheme 1):

Ethyl 7-bromo-4-hydroxy-2-naphthoate (1) [prepared as described in J. Org. Chem., 1996, 61, 4894-4912] is reacted with an appropriately substituted aryl- or heteroarylboronic acid with a catalyst such as PdC12-dppf to give ethyl 7-aryl- or 7-heteroaryl-4-hydroxy-2-naphthoate (2). This phenolic intermediate is then coupled with an appropriately substituted benzyl halide in the presence of a base such as potassium carbonate to provide intermediate 3. Alternatively, this phenolic intermediate can be reacted with an appropriately substituted benzyl alcohol in the presence of 1,1′-(azodicarbonyl)dipiperidine and a trialkylphosphine (Mitsunobu conditions) to provide intermediate 3. Hydrolysis of the ester 3 with aqueous sodium or lithium hydroxide in a mixture of tetrahydrofuran and methanol yields final product 4.

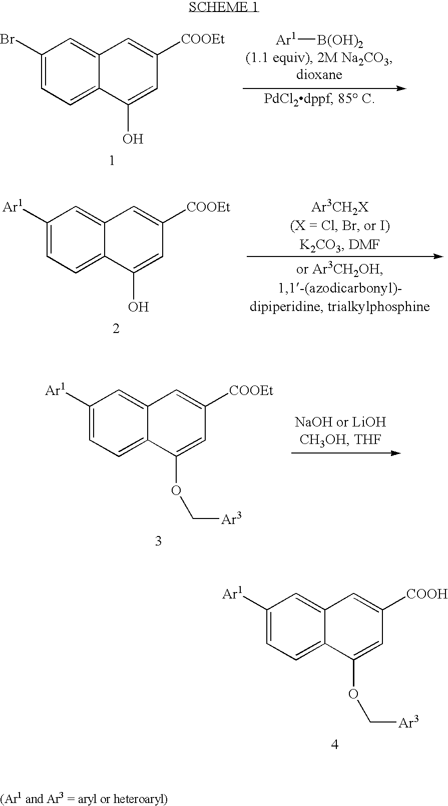

### Method B (Scheme 2):

Ethyl 7-benzyloxy-4-hydroxy-2-naphthoate (5) is reacted with trifluoromethanesulfonic anhydride (Tf₂O) and pyridine to produce the triflate intermediate 6 which can be coupled with an appropriately substituted aryl- or heteroarylboronic acid in the presence of a catalyst such as PdCl₂-dppf to produce the ethyl 7-benzyloxy-4-aryl- or 4-heteroaryl-2-naphthoate derivative 7. The benzyl group can be removed by hydrogenolysis in the presence of a palladium catalyst, such as palladium-on-carbon, to give the phenolic intermediate 8 which can be reacted as in Method A with an appropriately substituted benzyl halide to provide intermediate 9. The ester 9 is then hydrolysed as described in Method A using sodium or lithium hydroxide to yield final product 10. This method can also be readily adapted to combinatorial methods (as exemplified in Scheme 3) with the use of screw top test tubes as reaction vessels, potassium carbonate in triglyme for alkylation, lithium hydroxide for hydrolysis, formic acid for neutralization, and centrifugal evaporation to afford crude products which are purified using mass-directed preparative LC/MS.

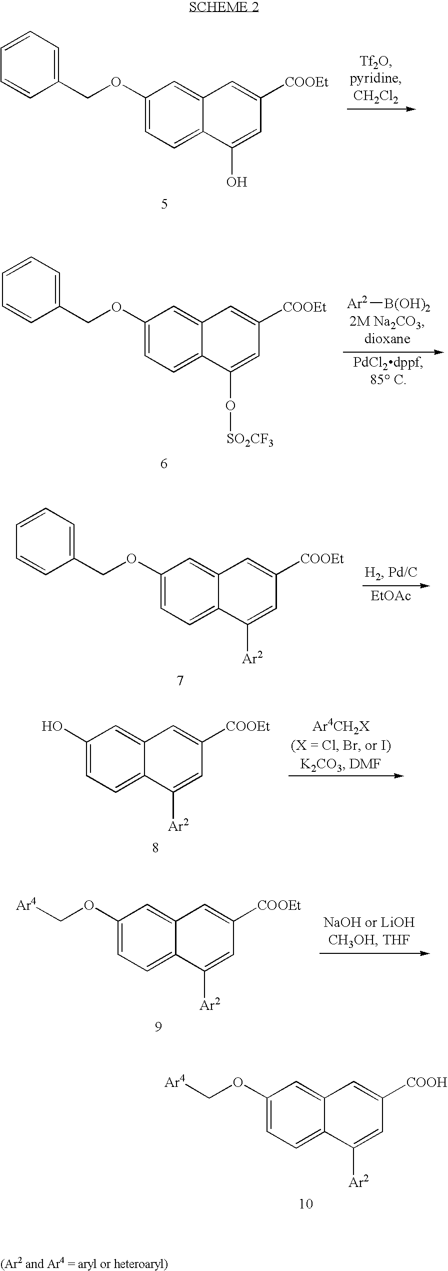

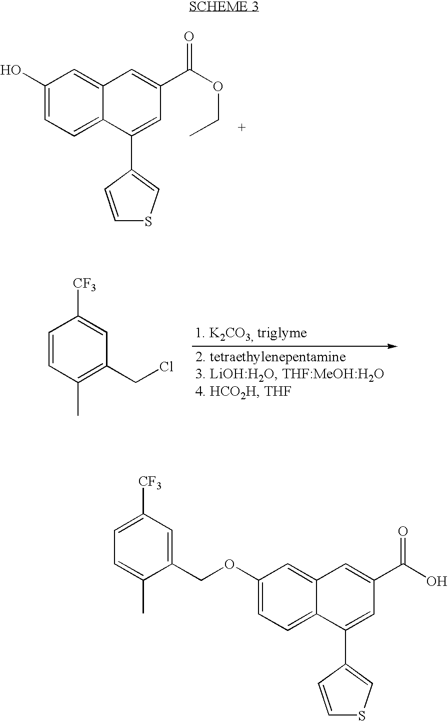

### Method C (Scheme 4):

The phenolic intermediate 2 is treated with Tf₂O and pyridine to give the triflate intermediate 11 which can be converted to ester 13 by cross-coupling with an appropriately substituted aryl- or heteroarylboronic acid and PdCl₂-dppf or, alternatively, can be converted to the boronate 12 using bis(pinacolato)diboron in the presence of PdC12-dppf. The conversion of 12 to 13 is carried out as described in Method A. The ester 13 is then hydrolysed as described in Method A using sodium or lithium hydroxide to yield final product 14.

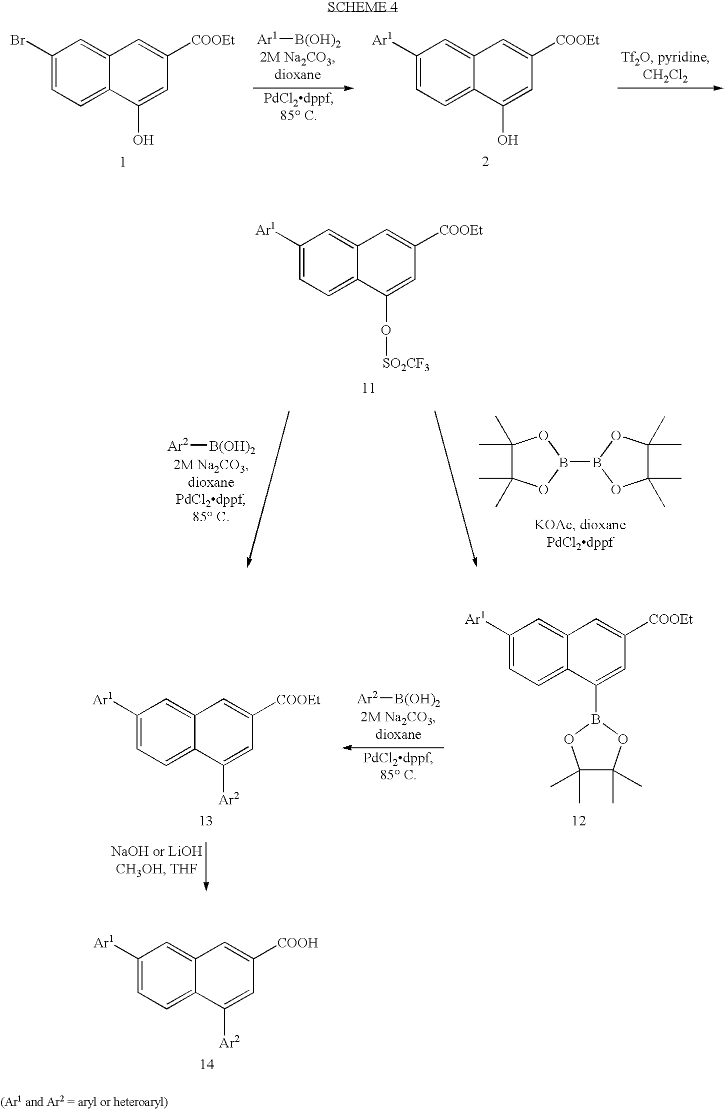

### Method D (Scheme 5):

The phenolic intermediate 1 is reacted with N-fluoropyridinium triflate in a solvent such as hot chlorobenzene to yield the 3-fluoro intermediate 15. The conversion of 15 to 19 is accomplished in a manner similar to the one described in Method C. The fluoro derivative 19 can be further treated with lithium hydroxide in hot DMSO to yield the phenolic derivative 20.

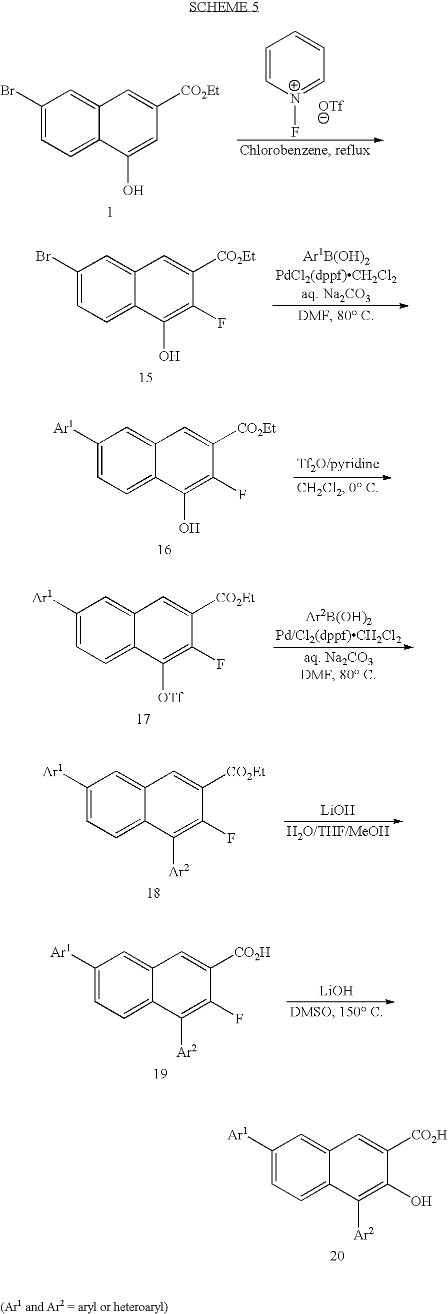

### Method E (Scheme 6):

Ethyl 7-bromo-4-hydroxy-2-naphthoate (1) is reacted as in Method A with an appropriately substituted benzyl halide to provide the intermediate bromide 21 which is then treated as in Method C with bis(pinacolato)diboron and a catalyst such as PdCl₂-dppf to produce the boronate intermediate 22. Treatment of 22 with hydrogen peroxide in methanol affords the 7-hydroxynaphthoate derivative 23 which is then converted to 24 in a sequence similar to the one described in Method A. Hydrolysis with sodium or lithium hydroxide provides the final product 25.

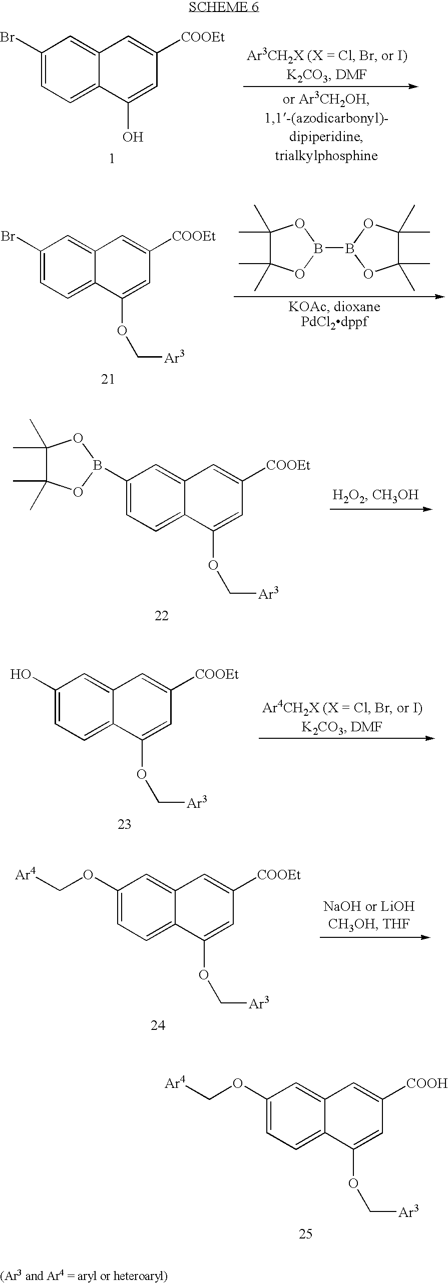

### Method F (Scheme 7):

Ethyl 7-benzyloxy-4-hydroxy-2-naphthoate (5), which is prepared in a 3-step sequence shown in Scheme 7 from 3-(benzyloxy)-benzaldehyde (26), is treated with Tf₂O and a base to afford intermediate 6 which is coupled with an appropriately substituted aryl- or heteroarylboronic acid and a catalyst such as PdCl₂-dppf to produce intermediate 7. Intermediate 7 is treated with carbon tetrabromide and an alcohol or with 1-propanethiol and aluminum chloride to afford 8 which is further processed to final product 14 as depicted in Scheme 7. This method and intermediate 8 can also be readily adapted to combinatorial preparative methods (as exemplified in Scheme 8) with the use of screw top test tubes as reaction vessels, formic acid for neutralization, and centrifugal evaporation to afford crude products which are purified using mass-directed preparative LC/MS.

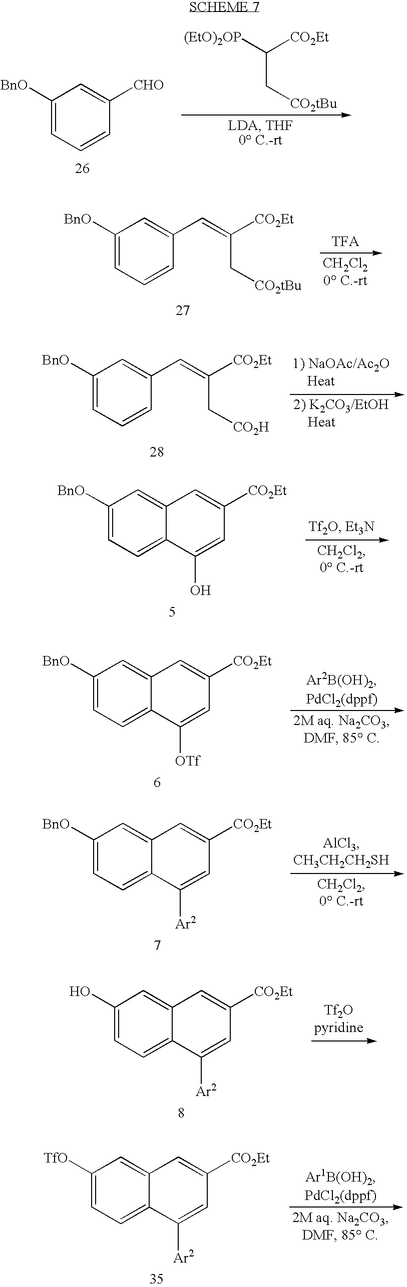

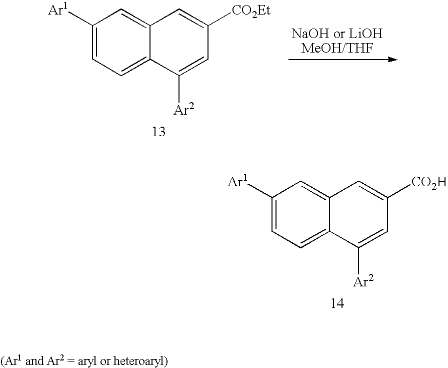

### Method G (Scheme 8):

4-Fluoro-3-methoxy-benzaldehyde (38) is treated with sodium thiomethoxide in DMF to provide the thioether derivative 39. Condensation of 39 with tert-butyl 3-ethoxycarbonyl-3-(phosphonodiethyl)propionate [prepared as described in Heterocyclic Commun., 9: 587-592 (2003)] with a base such as lithium N,N-diisopropylamide (LDA) followed by cleavage of the tert-butyl ester group provides acid intermediate 41. Cyclisation of 41 is accomplished in the presence of sodium acetate and acetic anhydride followed by treatment with potassium carbonate to yield 42. Conversion of 42 to 4-arylnaphthoate intermediate 44 is accomplished following methodologies described in Method A. Cleavage of the methyl ether 44 is effected with an alkanethiol and aluminum chloride which provides the phenolic intermediate 45 which is converted to the final product 48 using conditions described in Method A. Alternatively, 47 is treated with an oxidizing agent, such as hydrogen peroxide in the presence of sodium tungstate and a phase-transfer reagent, to provide the sulfinyl and sulfonyl derivatives 49 and 50. Conversion of 49 and 50 to the final carboxylic acids 51 and 52 is accomplished following procedures described in Method A.

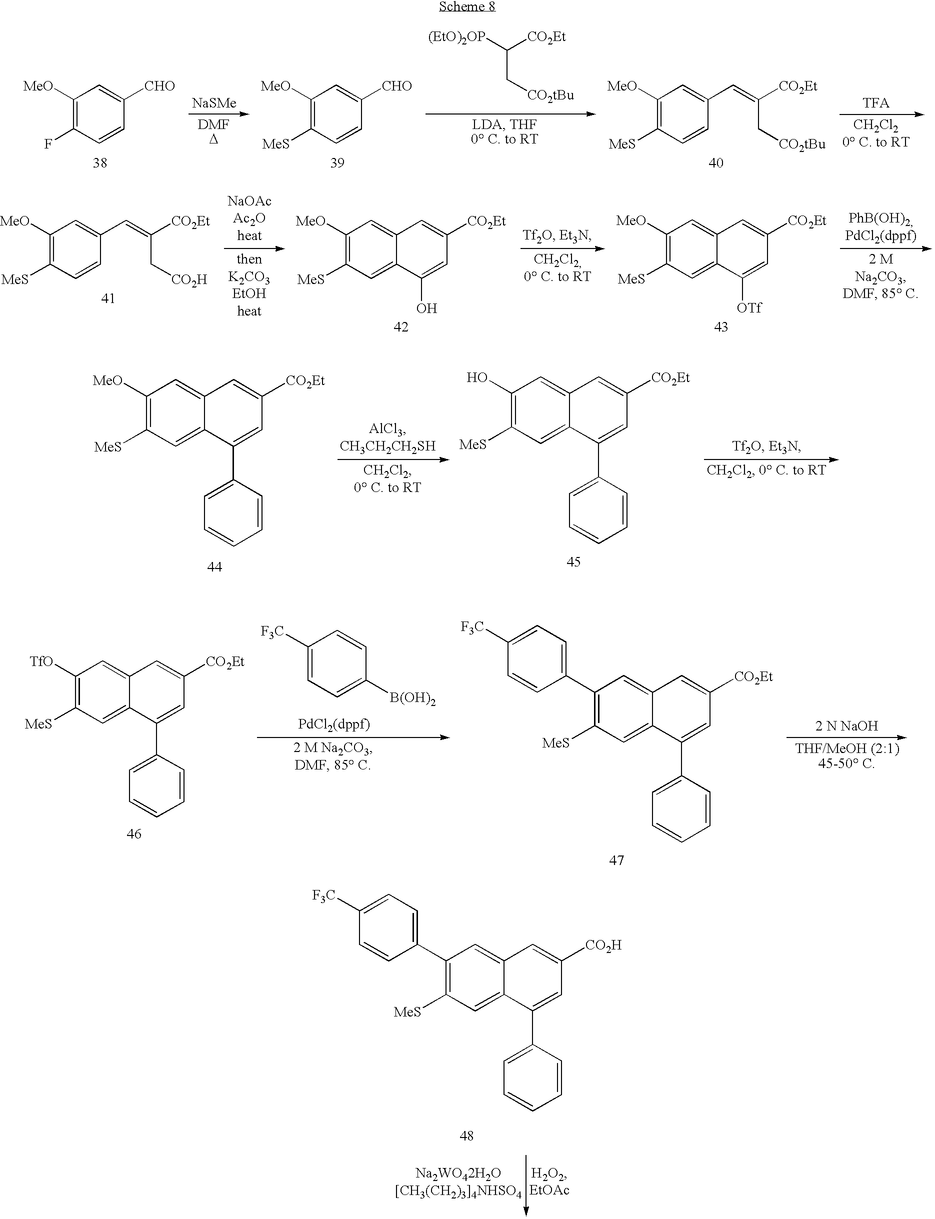

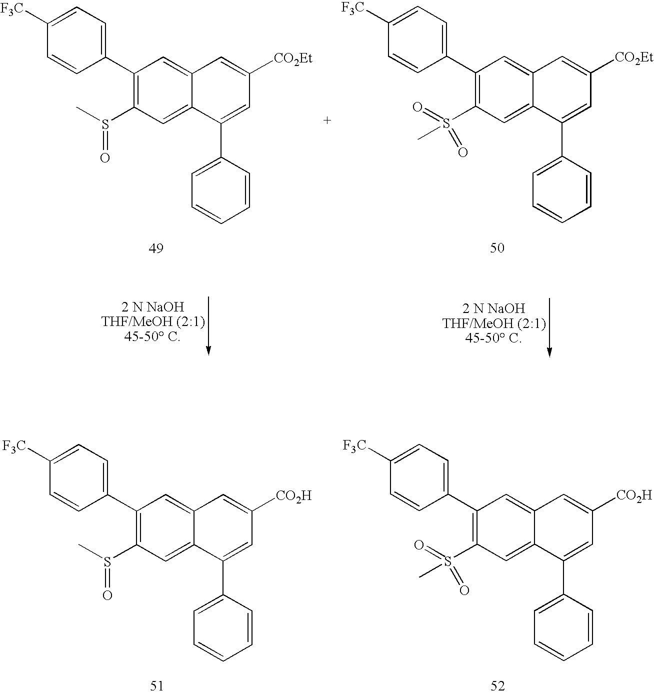

### Method H (Scheme 9):

Ethyl 7-benzyloxy-4-hydroxy-2-naphthoate 5 is treated with SEMC1 and a base to protect the hydroxy group at C-4. The phenol at C-7 is regenerated by catalytic hydrogenation with hydrogen and palladium on charcoal to afford intermediate 53 which is alkylated with an appropriately substituted benzyl halide, in a manner analogous to Method A, to afford intermediate 54. Intermediate 54 is converted into final products 55 or 56 using methods similar to those of Method B.

This method can also be readily adapted to combinatorial methods (as shown in Scheme 10) with the use of screw top test tubes as reaction vessels, palladium-tetrakis(triphenylphosphine) for coupling, lithium hydroxide for hydrolysis, formic acid for neutralization, and centrifugal evaporation to afford crude products which are purified using mass-directed preparative LC/MS.

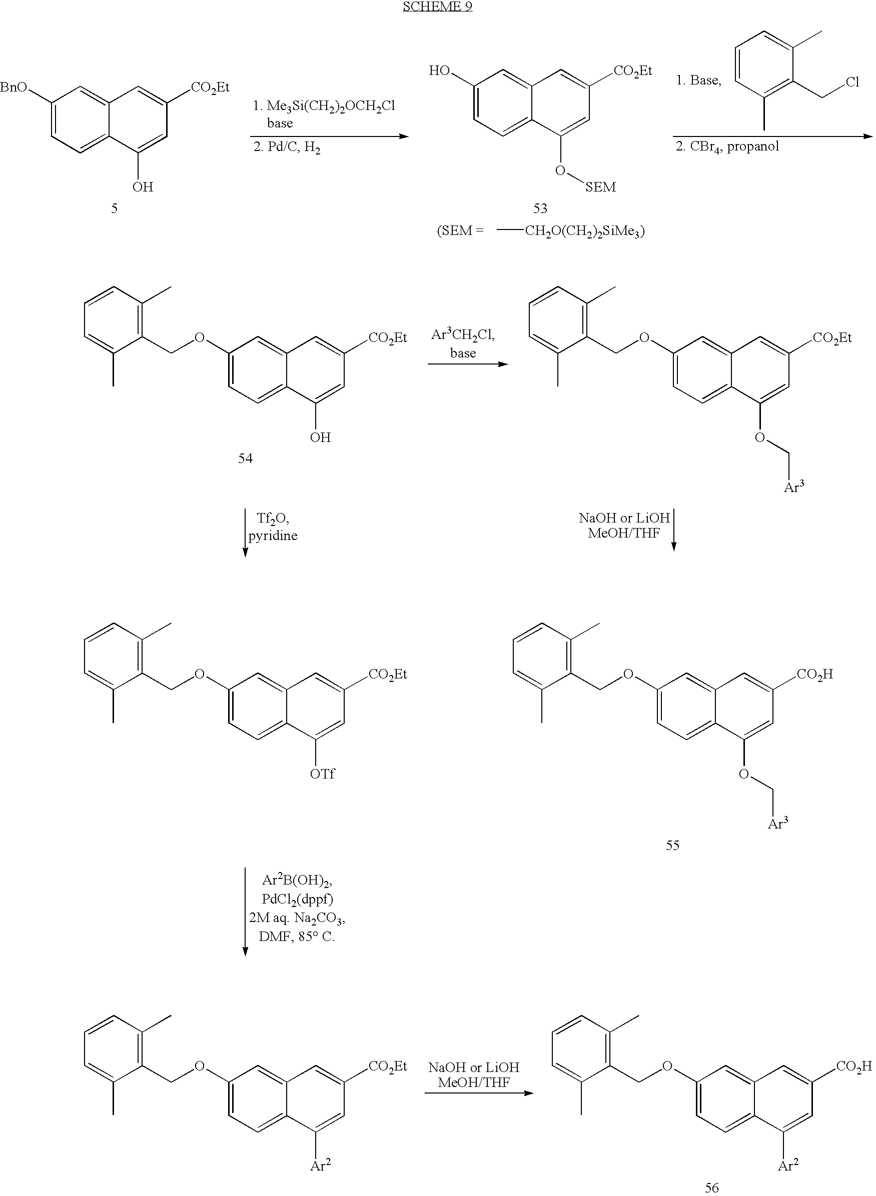

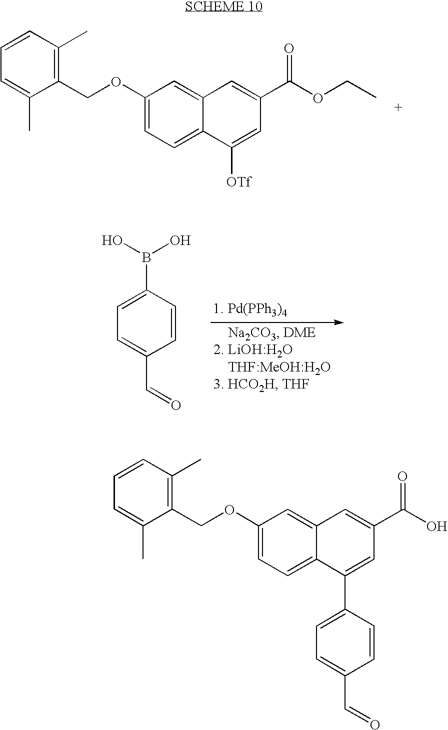

### Method I (Scheme 11):

A typical carboxylic acid prodrug can be prepared using an acid such as 4-{4-[1-(tert-butoxycarbonyl)piperidin-4-yl]phenyl}-7-[4-(trifluoromethyl)phenyl]-2-naphthoic acid 57 which is treated with 2-chloro-N,N-dimethylacetamide and a base to protect the carboxylic acid and afford 58. The free amine is regenerated using TFA. The methanesulfonic acid salt 59 can be prepared with a stoichiometric amount of methanesulfonic acid in a solvent such as dioxane.

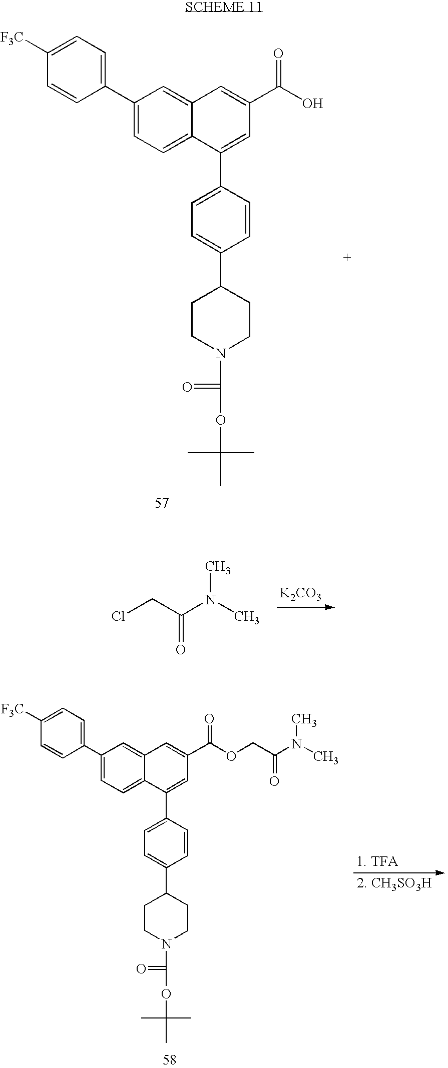

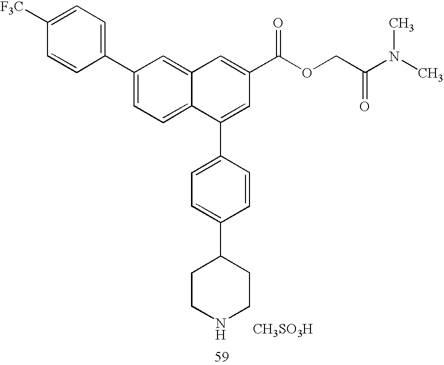

### Example 1

### 4-{4-[(1R)-2,2-Difluoro-1-hydroxyethyl]phenyl}-7-[4-(trifluoromethyl)phenyl]-2-naphthoic acid

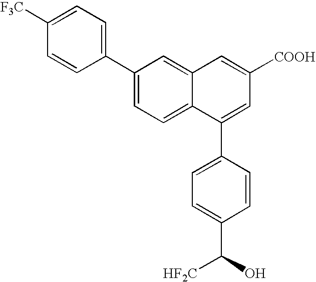

### Step 1

A suspension of ethyl 7-bromo-4-hydroxy-2-naphthoate (443 mg, 1.5 mmol), 4-(trifluoromethyl)benzeneboronic acid (313 mg, 1.650 mmol), DMF (4 mL) and 2M sodium carbonate (2.25 mL, 4.50 mmol) was degassed and PdCl₂(dppf) (21.95 mg, 0.030 mmol) was added. The mixture was heated at 85° C. for 3 h. It was cooled, diluted with EA and poured into water. It was extracted twice with EA and the combined EA layers were dried in the usual manner. Removal of the solvent gave a residue which was passed through on a short pad of SiO₂ eluting with EA:Hexanes (1:3) to give ethyl 4-hydroxy-7-[4-(trifluoromethyl)phenyl]-2-naphthoate. MS: M−H (−ESI)=359.1.

### Step 2

Trifluoromethanesulfonic anhydride (0.269 mL, 1.594 mmol) was added at −78° C. to a suspension of the phenol from Step 1 (430 mg, 1.386 mmol) and pyridine (0.168 mL, 2.079 mmol) in CH₂Cl₂ (7 mL). The mixture was warmed to RT and stirred for 3 h. It was then diluted with CH₂Cl₂ and washed with 10% aqueous NaHCO₃, 1N HCl, brine and dried with MgSO₄. Volatiles were removed and the residue triturated with hexanes to give ethyl 7-[4-(trifluoromethyl)phenyl]-4-{[(trifluoromethyl)sulfonyl]oxy}-2-naphthoate which was used in the next step without further purification.

¹H NMR (500 MHz, acetone-d₆): δ 9 (1H, d), 8.85-8.9 (1H, s), 8.65-8.7 (1H, s), 8.5-8.55 (1H, s), 8.1 (3H, m), 7.9 (2H, d), 4.5 (2H, q), 1.5 (12H, s), 1.45 (3H, t) ppm; MS: M-triflate (−ESI)=359.0

### Step 3: 1-(4-Bromophenyl)-2,2-difluoroethanone

To a cold (−78° C.) stirred solution of 1,4-dibromobenzene (86.4 g, 366 mmol) in tetrahydrofuran (800 mL) was added n-butyllithium (228 mL, 1.6 M in hexanes, 366 mmol). The mixture was stirred at −78° C. for 30 min and ethyl difluoroacetate (50 g, 402 mmol) was added over 2 min. The mixture was stirred at −78 C for 1 h. The reaction was quenched with 1 N hydrochloric acid (250 mL) and allowed to attain room temperature. Methyl tert-butyl ether (250 mL) was added and the layers were separated. The organic layer was washed with brine (100 mL), dried (MgSO₄) and concentrated under reduced pressure. The residue was distilled under vacuum to give the difluoroketone as a white glassy solid which was used in the next step.

### Step 4: (1R)-1-(4-Bromophenyl)-2,2-difluoroethanol

The ketone prepared in step 3 (2.35 g, 10 mmoles) and commercial R-Alpine Borane (3.1 g, 12 mmol) were mixed together at room temperature and stirred for four d with some gas evolution. The reaction was cooled to 0° C. and acetaldehyde (168 μL, 3 mmol) was added. The bath was removed and stirring was continued at room temperature for 30 min Diethyl ether (20 mL) was added followed by ethanolamine (725 μL, 12 mmol). The mixture was stirred at room temperature for one h. The precipitate was removed by filtration and washed with pentane. The filtrate was concentrated under reduced pressure and purified by flash chromatography (90% hexanes:10% ethyl acetate to 70% hexanes:30% ethyl acetate) to give the desired material as a colorless oil.

1H NMR (500 MHz, acetone-d₆): δ 7.6 (2H, d), 7.45 (2H, d), 5.8-6.1 (1H, m), 5.4 (1H, d), 4.85-5.0 (1H, m) ppm.

### Step 5: (1R)-2,2-Difluoro-1-[4-(4,4,5,5-tetramethyl-1,3,2-dioxaborolan-2-yl)phenyl]ethanol

A mixture of the bromide from Step 4 (8 g, 33.7 mmol), potassium acetate (9.94 g, 101 mmol) and bis(pinacolato)diboron (10.28 g, 40.5 mmol) in DMF was degassed for 10 min and PdCl2(dppf) (1.235 g, 1.687 mmol) was added. The mixture was then stirred at 85° C. for 3 h. The mixture was cooled, water and Et₂O were added and the mixture was filtered on a pad of celite. The filtrate was extracted three times with diethyl ether. The combined organic fractions were washed with water then brine, dried with MgSO₄, filtered and the solvent was evaporated under reduced pressure. The residue was purified by chromatography on CombiFlash using Silicycle™ 230-400 mesh, eluting with EtOAc/hexanes (20%) to give the title compound which was used in the next step without further purification.

1H NMR (500 MHz, acetone-d₆): δ 7.8 (2H, d), 7.5 (2H, d), 5.8-6.1 (1H, m), 5.3 (1H, d), 4.85-4.95 (1H, m), 1.35 (12H, s) ppm.

### Step 6

A mixture of the triflate from Step 2 (570 mg, 1.158 mmol), (1R)-2,2-difluoro-1-[4-(4,4,5,5-tetramethyl-1,3,2-dioxaborolan-2-yl)phenyl]ethanol (428 mg, 1.505 mmol), DMF (6 mL) and 2M Na₂CO₃ (1.736 mL, 3.47 mmol) was degassed and PdCl₂(dppf)-CH₂Cl₂ (47.3 mg, 0.058 mmol) was added. The mixture was heated at 85° C. for 3 h. It was cooled, diluted with EA and washed with aqueous NaHCO₃ and brine. The organic layer was dried as usual and the volatiles removed. The residue was subjected to chromatography on SiO₂ using EA:H as eluant (1:5 to 1:3) to give ethyl 4-{4-[(1R)-2,2-difluoro-1-hydroxyethyl]phenyl}-7-[4-(trifluoromethyl)phenyl]-2-naphthoate.

### Step 7

A mixture of the ester from Step 6 (380 mg, 0.759 mmol), 2N LiOH (1.139 mL, 2.278 mmol), THF (3 mL) and MeOH (1 mL) was stirred at RT for 16 h. Most of the solvent was removed, and water followed by EA was added. The mixture was acidified with 1N HCl to pH about 4 and extracted twice with EA. The combined EA layers were washed with brine and dried. The residue was triturated in MTBE/Hexanes to give 4-{4-[(1R)-2,2-difluoro-1-hydroxyethyl]phenyl}-7-[4-(trifluoromethyl)phenyl]-2-naphthoic acid.

¹H NMR (500 MHz, acetone-d₆): δ 8.85 (1H, s), 8.65 (1H, s), 8.05-8.15 (5H, m), 7.9 (2H, d), 7.75 (2H, d), 7.65 (2H, d), 5.9-6.25 (1H, m), 5.4 (1H, OH), 5.0-5.1 (1H, m) ppm.

MS: M−H (−ESI)=471.0; [α]_{D}²⁵=−14.2° (c=1, CD₃COCD₃).

### Example 2

### 4-[5-(Methylsulfonyl)-3-thienyl]-7-[4-(trifluoromethyl)phenyl]-2-naphthoic acid

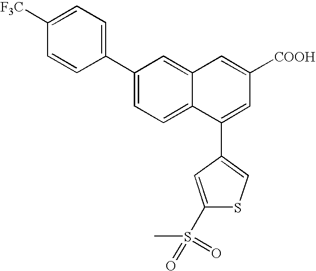

### Step 1

n-Butyllithium (1198 μl, 3.15 mmol) was added at −70° C. to a mixture of 2,4-dibromothiophene (726 mg, 3 mmol) in Et₂O (7500 μL) and the mixture was stirred for 10 min. An Et₂O (2 mL) solution of dimethyl disulfide (320 μl, 3.60 mmol) was then added dropwise. The mixture was stirred for 15 min at −70° C. and then warmed to 0° C. for 1 h. To the mixture was added dilute aqueous NH₄Cl and it was extracted twice with MTBE, dried as usual and the solvent was removed under vacuum. The residue was dissolved in EA (15 mL) and cooled to 0° C. Tetrabutylammonium hydrogen sulfate (50.9 mg, 0.150 mmol), sodium tungstate dihydrate (49.5 mg, 0.150 mmol) were added followed by 30% hydrogen peroxide (657 μL, 7.50 mmol). The mixture was stirred ON at 5° C. It was then diluted with EA and washed with dilute aqueous NaHSO₃, NaHCO₃ and dried as usual. The residue was purified by chromatography on SiO₂ using 1:3 EA:H as eluant to yield 4-bromo-2-methylsulfonyl-thiophene.

¹H NMR (500 MHz, acetone-d₆): δ 8.05 (1H, s), 7.8 (1H, s), 3.35 (3H, s) ppm.

### Step 2

PdCl₂(dppf)-CH₂Cl₂ adduct (20.42 mg, 0.025 mmol) was added to a degassed suspension of bis(pinacolato)diboron (133 mg, 0.525 mmol), 4-bromo-2-methylsulfonyl-thiophene and potassium acetate (147 mg, 1.500 mmol) in DMF (3 mL). The mixture was heated at 85-95° C. for 3 h. It was cooled to RT and ethyl 7-[4-(trifluoromethyl)phenyl]-4-{[(trifluoromethyl)sulfonyl]oxy}-2-naphthoate from Step 2, Example 1, (246 mg, 0.5 mmol) was added, followed by 2M Na₂CO₃ (0.750 mL, 1.500 mmol). The mixture was degassed again and then warmed to 85° C. for 3 h. It was cooled, diluted with EA and washed with saturated aqueous NaHCO₃, 1N HCl, brine and dried. The residue, after evaporation of the solvent, was passed through a short pad of SiO₂ eluting with 1:2 EA:H to give ethyl 4-[5-(methylsulfonyl)-3-thienyl]-7-[4-(trifluoromethyl)phenyl]-2-naphthoate.

### Step 3

2N LiOH (0.384 mL, 0.767 mmol) was added to the ester from Step 2 (129 mg, 0.256 mmol) in THF (2 mL) and MeOH (1 mL) and the mixture was stirred at RT overnight. Most of the solvent was removed by evaporation under diminished pressure, and the residue was diluted with water. It was acidified with 1N HCl and extracted twice with EA (using a little THF). The organic layer was washed with brine and dried as usual. The residue was triturated with Et₂O, filtered and dried to give 4-[5-(methylsulfonyl)-3-thienyl]-7-[4-(trifluoromethyl)phenyl]-2-naphthoic acid. MS: M+H (+ESI)=475.1.

### Example 3

### 4-[4-(2,2,2-Trifluoro-1-hydroxyethyl)phenyl]-7-[4-(trifluoromethyl)phenyl]-2-naphthoic acid

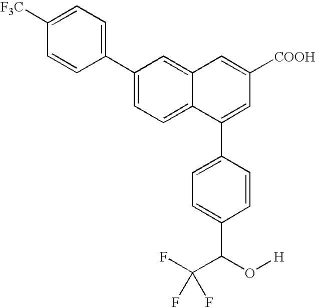

### Step 1

A suspension of ethyl 7-[4-(trifluoromethyl)phenyl]-4-{[(trifluoromethyl)sulfonyl]oxy}-2-naphthoate (394 mg, 0.8 mmol) as prepared in Example 1, Step 2, bis(pinacolato)diboron (223 mg, 0.880 mmol), dioxane (5 ml) and potassium acetate (236 mg, 2.400 mmol) was degassed and PdCl₂(dppf)-CH₂Cl₂ adduct (16.33 mg, 0.020 mmol) was added. The mixture was heated at 85° C. for 4 h. It was cooled and most of the dioxane was removed under vacuum. The residue was purified on a short pad of silica gel eluting with 1:6 EA:H to give ethyl 4-(4,4,5,5-tetramethyl-1,3,2-dioxaborolan-2-yl)-7-[4-(trifluoromethyl)phenyl]-2-naphthoate. MS: M+H (+ESI)=471.1

### Step 2

A mixture of ethyl 4-(4,4,5,5-tetramethyl-1,3,2-dioxaborolan-2-yl)-7-[4-(trifluoromethyl)phenyl]-2-naphthoate (384 mg, 0.817 mmol) as prepared in Step 1, 1-(4-bromophenyl)-2,2,2-trifluoroethanone (227 mg, 0.898 mmol), Na₂CO₃ (1.225 mL, 2.450 mmol) and DMF (5 mL) was degassed. PdCl₂(dppf)-CH₂Cl₂ adduct (33.3 mg, 0.041 mmol) was added and the mixture was heated at 85° C. for 4 h. It was cooled, diluted with EA and poured onto dilute aqueous NaHCO₃. It was extracted twice with EA and the organic layer was dried in the usual manner. The crude product was dissolved in THF (5 mL) and methanol (2 mL) and cooled to ° C. Sodium borohydride (30.9 mg, 0.817 mmol) was added and the mixture was reacted overnight at RT. It was poured into water and extracted twice with EA, washed with brine and dried. The residue was subjected to chromatography on SiO₂ using EA:H (1:5) as eluant to give ethyl 4-[4-(2,2,2-trifluoro-1-hydroxyethyl)phenyl]-7-[4-(trifluoromethyl)phenyl]-2-naphthoate.

### Step 3

A mixture of the ester from Step 2 (277 mg, 0.534 mmol) and 2N LiOH (0.534 mL, 1.069 mmol) in THF (4 mL) and MeOH (1 mL) was stirred overnight at RT. Most of the solvent was removed by evaporation under diminished pressure and the residue was diluted with water. It was acidified with 1N HCl and extracted twice with EA. The combined organic layers were dried as usual. The residue was subjected to chromatography on SiO₂ using EA:H:acetic acid (1:2:0.01) as eluant to give 4-[4-(2,2,2-trifluoro-1-hydroxyethyl)phenyl]-7-[4-(trifluoromethyl)phenyl]-2-naphthoic acid. MS: M−H (−ESI)=489.0.

### Example 4

### 4-{4-[(1R)-2,2,2-Trifluoro-1-hydroxyethyl]phenyl}-7-[4-(trifluoromethyl)phenyl]-2-naphthoic acid

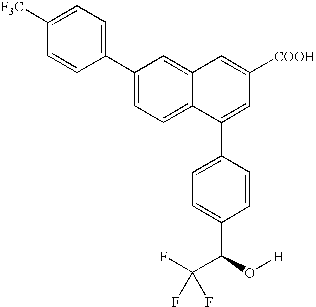

### and 4-{4-[(1S)-2,2,2-Trifluoro-1-hydroxyethyl]phenyl}-7-[4-(trifluoromethyl)phenyl]-2-naphthoic acid

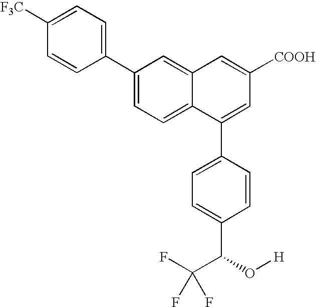

The racemic mixture of 4-[4-(2,2,2-trifluoro-1-hydroxyethyl)phenyl]-7-[4-(trifluoromethyl)phenyl]-2-naphthoic acid described in Example 3 was separated by chiral HPLC (Chiralpak AD, 20% iPrOH/hexanes+0.25% formic acid at 1 mL/min) to afford 4-{4-[(1R)-2,2,2-trifluoro-1-hydroxyethyl]phenyl}-7-[4-(trifluoromethyl)phenyl]-2-naphthoic acid (retention time 9.74 min) [MS: M−H (−ESI)=489.0] and 4-{4-[(1S)-2,2,2-trifluoro-1-hydroxyethyl]phenyl}-7-[4-(trifluoromethyl)phenyl]-2-naphthoic acid (retention time 7.32 min).

MS: [M−H (−ESI)=489.0].

### Example 5

### 4-[4-(Methylsulfinyl)phenyl]-7-[4-(trifluoromethyl)phenyl]-2-naphthoic acid

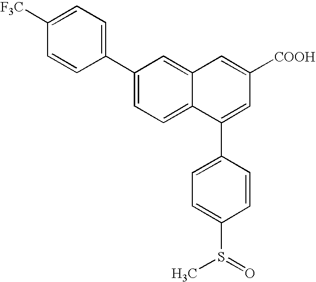

### Step 1

A suspension of 1-bromo-4-(methylsulfinyl)benzene (5.48 g, 25 mmol), bis(pinacolato)diboron (6.98 g, 27.5 mmol), dioxane (100 mL), potassium acetate (7.36 g, 75 mmol) was degassed and PdCl₂(dppf)-CH₂Cl₂ adduct (0.204 g, 0.250 mmol) was added. The mixture was heated at 85° C. for 4 h. It was cooled and most of the solvent was removed by evaporation under diminished pressure. The residue was passed through a short pad of SiO₂ eluting with 2:1 EA:H to give 2-(4-bromophenyl)-4,4,5,5-tetramethyl-1,3,2-dioxaborolane.

¹H NMR (500 MHz, acetone-d₆): δ 7.9 (2H, d), 7.7 (2H, d), 2.75 (3H, s), 1.4 (12H, s) ppm.

### Step 2

A suspension of ethyl 7-[4-(trifluoromethyl)phenyl]-4-{[(trifluoromethyl)sulfonyl]oxy}-2-naphthoate (320 mg, 0.65 mmol) from Example 1, Step 2, the boronate from Step 1 (190 mg, 0.715 mmol), DMF (8 mL) and Na₂CO₃ (0.975 mL, 1.950 mmol) was degassed. PdCl₂(dppf)-CH₂Cl₂ adduct (53.1 mg, 0.065 mmol) was added and the mixture was heated at 85° C. for 4 h. It was cooled and poured into dilute aqueous NH₄Cl. The mixture was extracted twice with EA and the combined organic layers were dried in the ususal manner. The residue from evaporation was passed through a short pad of SiO₂ eluting with 2:1 EA:Hexanes to give ethyl 4-[4-(methylsulfinyl)phenyl]-7-[4-(trifluoromethyl)phenyl]-2-naphthoate.

¹H NMR (500 MHz, acetone-d₆): δ 8.85 (1H, s), 8.65 (1H, s), 8.15 (2H, d), 8.0-8.1 (3H, m), 7.9-8.0 (4H, m), 7.8 (2H, d), 4.45-4.5 (2H, q), 2.85 (3H, s), 1.45 (3H, t) ppm.

### Step 3

A mixture of the ester from Step 2 (205 mg, 0.425 mmol), 2N LiOH (0.637 mL, 1.275 mmol) in THF (3 mL) and MeOH (1 mL) was stirred overnight at RT. Most of the solvent was removed and the residue was diluted with water. It was acidified with 1N HCl, extracted twice with EA and dried as usual. After removal of the solvent, the residue was triturated with MTBE to give 4-[4-(methylsulfinyl)phenyl]-7-[4-(trifluoromethyl)phenyl]-2-naphthoic acid.

MS: M−H (−ESI)=453.1.

### Example 6

### 4-{4-[(1R)-2,2-Difluoro-1-hydroxyethyl]phenyl}-7-[4-(methylthio)phenyl]-2-naphthoic acid

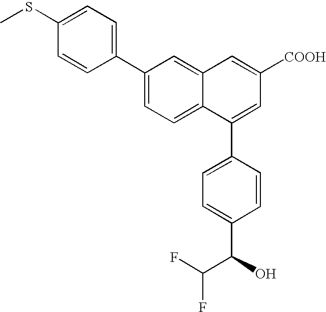

### Step 1

A suspension of ethyl 7-bromo-4-hydroxy-2-naphthoate (5.90 g, 20 mmol), 4-(methylthio)benzeneboronic acid (4.03 g, 24.00 mmol), DMF (100 mL) and Na₂CO₃ (30.0 mL, 60.0 mmol) was degassed then PdCl₂(dppf) (0.732 g, 1.000 mmol) was added. The mixture was stirred at 75° C. for 3 h. It was cooled and diluted with EA and poured into dilute NH₄OH. It was extracted twice with EA and the combined organic extracts were dried as usual. Removal of the solvent gave a residue which was triturated with MTBE to give ethyl 4-hydroxy-7-[4-(methylthio)phenyl]-2-naphthoate.

¹H NMR (500 MHz, acetone-d₆): δ 9.4 (1H, s), 8.4 (1H, d), 8.3 (1H, s), 8.25 (1H, s), 7.95 (1H, d), 7.8 (2H, d), 7.5 (1H, s), 7.4 (2H, d), 4.4 (2H, q), 2.6 (3H, s), 1.4 (3H, t) ppm.

### Step 2

N-phenyl bis(trifluoromethanesulfonimide) (583 mg, 1.632 mmol) was added as a CH₂Cl₂ solution (5 mL) to a mixture of the phenol from Step 1 at −78° C. (502 mg, 1.483 mmol), Et₃N (0.310 mL, 2.225 mmol) and DMAP (9.06 mg, 0.074 mmol) in 1,2-dichloroethane (10 mL) and DMF (2 mL). The mixture was warmed to RT and stirred for 2 h. It was diluted with CH₂Cl₂ and poured into aqueous NH₄Cl and extracted twice. The combined organic layers were washed with brine and dried in the usual manner. The residue was passed through a short pad of SiO₂ eluting with 1:5 EA:H to give ethyl 7-[4-(methylthio)phenyl]-4-{[(trifluoromethyl)-sulfonyl]oxy}-2-naphthoate which was used in the next step without further purification.

¹H NMR (500 MHz, acetone-4): δ 8.9 (1H, d), 8.6 (1H, s), 8.2-8.35 (2H, d), 8.1 (1H, s), 7.9 (1H, s), 7.5 (2H, d), 4.5 (2H, q), 2.6 (3H, s), 1.45 (3H, t) ppm.

### Step 3

To a degassed suspension of ethyl 7-[4-(methylthio)phenyl]-4-{[(trifluoromethyl)sulfonyl]oxy}-2-naphthoate (235 mg, 0.5 mmol), (1R)-2,2-difluoro-1-[4-(4,4,5,5-tetramethyl-1,3,2-dioxaborolan-2-yl)phenyl]ethanol (284 mg, 1.000 mmol), 2M Na₂CO₃ (0.750 mL, 1.500 mmol) and DMF (4 mL) was added PdCl₂(dppf) (36.6 mg, 0.050 mmol) and the mixture was reacted for 4 h at 80° C. It was cooled and diluted with EA. The mixture was extracted twice with EA and the combined organic layers were washed with 1N HCl, brine and dried in the usual manner. After evaporation, the residue was subjected to chromatography on SiO₂ using 1:3 EA:H as eluant to give ethyl 4-{4-[(1R)-2,2-difluoro-1-hydroxyethyl]phenyl}-7-[4-(methylthio)phenyl]-2-naphthoate.

¹H NMR (500 MHz, acetone-d₆): δ 8.75 (1H, s), 8.5 (1H, s), 8.0 (2H, m), 7.85 (2H, d), 7.75 (2H, d), 7.6 (2H, d), 7.45 (2H, d), 6.1 (1H, m), 5.45 (1H, OH), 5.05 (1H, m), 4.45 (2H, m), 2.6 (3H, s), 1.45 (3H, t) ppm.

### Step 4

To a solution of the ester from Step 3 (100 mg, 0.209 mmol) in THF (3 mL) and MeOH (1 mL) was added 2N NaOH (0.418 mL, 0.836 mmol) and the reaction was warmed to 80° C. for 2 h. It was cooled and most of the solvent was removed by evaporation under diminished pressure. The residue was diluted with water, acidified to pH about 3 with 1N HCl and extracted twice with EA. After drying and removal of the solvent, the residue was triturated with MTBE, the resulting solid filtered and dried to give 4-{4-[(1R)-2,2-difluoro-1-hydroxyethyl]phenyl}-7-[4-(methylthio)phenyl]-2-naphthoic acid. MS: M−H (−ESI)=449.0.

### Example 7

### 4-{4-[(1R)-2,2-Difluoro-1-hydroxyethyl]phenyl}-7-(4-fluorophenyl)-2-naphthoic acid

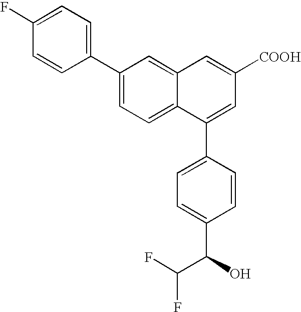

### Step 1

The intermediate phenol was prepared as in Example 1, Step 1, but using 4-fluorobenzeneboronic acid to give ethyl 7-(4-fluorophenyl)-4-hydroxy-2-naphthoate.

¹H NMR (500 MHz, acetone-d₆): δ 9.45 (1H, OH), 8.4 (1H, d), 8.25-8.35 (2H, d), 7.85-8.0 (3H, m), 7.55 (1H, s), 7.3 (2H, m), 4.4 (2H, m), 1.4 (3H, m) ppm.

### Step 2

The intermediate triflate was prepared as in Example 1, Step 2 but using the above phenol to give ethyl 7-(4-fluorophenyl)-4-{[(trifluoromethyl)sulfonyl]oxy}-2-naphthoate which was used in Step 3 without further purification.

### Step 3

PdCl₂(dppf)-CH₂Cl₂ adduct (30.6 mg, 0.038 mmol) was added to a degassed mixture of the triflate from Step 2 (332 mg, 0.75 mmol), bis(pinacolato)diboron (200 mg, 0.788 mmol) and potassium acetate (221 mg, 2.250 mmol) in DMF (5 mL). The mixture was heated at 85° C. for 2 h and then cooled. Aqueous 2M Na₂CO₃ (1.125 mL, 2.250 mmol) and a solution of (1R)-1-(4-bromophenyl)-2,2-difluoroethanol (178 mg, 0.750 mmol) in DMF (1 mL) were added. The mixture was degassed again and PdCl₂(dppf)-CH₂Cl₂ adduct was added. The mixture was heated at 85° C. for 3 h. It was cooled, diluted with water and extracted twice with EA. The combined organic layers were washed with aqueous NaHCO₃, brine and dried as usual. After removal of the solvent, the residue was passed through SiO₂ eluting with 1:10 EA:Toluene to give a product which was resubjected to chromatography on SiO₂ using 1:5 EA:H as eluant to give ethyl 4-{4-[(1R)-2,2-difluoro-1-hydroxyethyl]phenyl}-7-(4-fluorophenyl)-2-naphthoic acid.

MS: M+H (+ESI)=451.0.

### Step 4

2N LiOH (0.977 mL, 1.954 mmol) was added to a solution of the ester from Step 3 (176 mg, 0.391 mmol) in THF (3 mL) and MeOH (1 mL). The mixture was warmed to 75° C. for 3 h. It was cooled and most of the solvent was removed. The residue was diluted with water, acidified with 1N HCl, and extracted twice with EA. The combined organic layers were dried as usual and the residue after evaporation was triturated with Et₂O to give 4-{4-[(1R)-2,2-difluoro-1-hydroxyethyl]phenyl}-7-(4-fluorophenyl)-2-naphthoic acid.

¹H NMR (500 MHz, acetone-4): δ 8.8 (1H, s), 8.5 (1H, s), 7.9-8.1 (5H, m), 7.75 (2H, d), 7.6 (2H, d), 7.3 (2H, m), 6.1 (1H, m), 5.45 (1H, OH), 5.05 (1H, m) ppm; MS: M−H (−ESI)=412.1.

### Example 8

### 4-[5-(Methylsulfonyl)-3-thienyl]-7-[4-(trifluoromethoxy)phenyl]-2-naphthoic acid

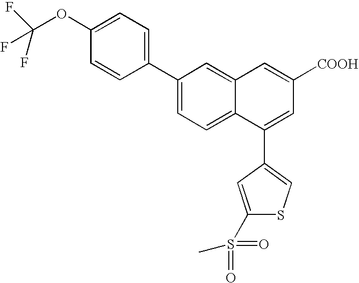

### Step 1

The first intermediate was prepared as in Example 6, Step 1, but using 4-(trifluoromethoxy)benzeneboronic acid and ethyl 7-bromo-4-hydroxy-2-naphthoate to give the intermediate phenol. The phenol was converted to the trifluoromethanesulfonate derivative as in Example 6, Step 2.

¹H NMR (CDCl₃): δ 8.72 (s, 1H), 8.26-8.20 (m, 2H), 8.07 (s, 1H), 8.02-7.98 (m, 1H), 7.76 (d, 2H), 7.40 (d, 2H), 7.29 (s, 2H), 4.51 (q, 2 μl), 1.50 (t, 3H) ppm.

### Step 2

The triflate was coupled with 4-bromo-2-methylsulfonyl-thiophene (Example 2, Step 1) as in Example 7, Step 3 to give ethyl 4-[5-(methylsulfonyl)-3-thienyl]-7-[4-(trifluoromethoxy)phenyl]-2-naphthoate.

1H NMR (500 MHz, acetone-4): δ 8.8 (1H, s), 8.55 (1H, s), 8.25 (1H, s), 8.15 (1H, d), 8.0-8.15 (5H, m), 7.55 (2H, d), 4.5 (2H, m), 3.4 (3H, s), 1.45 (3H, t) ppm.

### Step 3

2N LiOH (0.527 mL, 1.055 mmol) was added to the ester from Step 2 (183 mg, 0.352 mmol) in THF (4 mL) and MeOH (1 mL). The mixture was stirred at 70° C. for 2 h. It was cooled, most of the solvent removed, and the residue was diluted with water. It was acidified to pH about 3 with 1N HCl and extracted twice with EA. The combined organic layers were washed with brine and dried as usual. After removal of the solvent, the residue was triturated with MTBE to give 4-[5-(methylsulfonyl)-3-thienyl]-7-[4-(trifluoromethoxy)phenyl]-2-naphthoic acid. MS: M−H (−ESI)=491.2.

### Example 9

### 4-[5-(2,2-Difluoro-1-hydroxyethyl)-3-thienyl]-7-[4-(trifluoromethyl)phenyl]-2-naphthoic acid

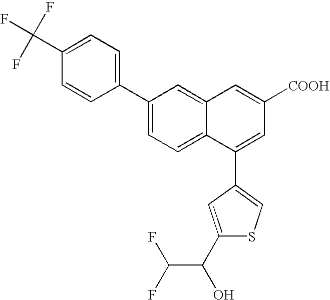

### Step 1: 1-(4-Bromo-2-thienyl)-2,2-difluoroethanol

n-Butyllithium (2.50M, 18.58 mL, 46.4 mmol) was added at −78° C. to a stirred mixture of 2,4-dibromothiophene (5 mL, 44.2 mmol) in diethyl ether (85 mL) and the mixture was stirred at −78° C. for 10 min. This mixture was then added to ethyl difluoroacetate (4.64 mL, 46.4 mmol) in Et₂O (25 mL) at −78° C. and the resulting mixture was stirred at that temperature for 1 h. The mixture was warmed up to room temperature, hydrochloric acid (1 M) was added and the mixture was extracted twice with diethyl ether (50 mL). The combined organic fractions were washed with brine (50 mL), dried (MgSO₄), filtered and the solvent was evaporated under reduced pressure to afford 1-(4-bromo-2-thienyl)-2,2-difluoroethanone as a yellow oil which was used in the next step without further purification. MS: M−H (−ESI)=238.8, 240.8.

### Step 2

Sodium borohydride (0.718 g, 18.98 mmol) was added at 0° C. to a stirred mixture of 1-(4-bromo-2-thienyl)-2,2-difluoroethanone (4.16 g, 17.26 mmol) in methanol and the mixture was stirred at 0° C. for 30 min. The mixture was diluted with Et₂O and hydrochloric acid (1 M, 25 mL) was added. The aqueous phase was extracted twice with diethyl ether (75 mL). The combined organic fractions were washed with saturated brine solution (50 mL), dried (MgSO₄), filtered and the solvent was evaporated under reduced pressure. The residue was purified by column chromatography on silica gel (120 g), eluting with EtOAc/hexanes (0-30%) to give 1-(4-bromo-2-thienyl)-2,2-difluoroethanol as a colorless solid.

¹H NMR (500 MHz, acetone-d₆): δ 7.55 (d, 1H), 7.14 (s, 1H), 6.01 (td, 1H), 5.82 (d, 1H), 5.24-5.16 (m, 1H) ppm.

### Step 3

A mixture of 1-(4-bromo-2-thienyl)-2,2-difluoroethanol (131 mg, 0.539 mmol) from Step 2, potassium acetate (132 mg, 1.346 mmol), bis(pinacolato)diboron (148 mg, 0.583 mmol) in DMF (3.5 mL) was degassed and PdCl₂(dppf).CH₂Cl₂ (17 mg, 0.023 mmol) was added. The mixture was heated to 85° C. for 2 h. It was cooled and ethyl 7-[4-(trifluoromethyl)phenyl]-4-{[(trifluoromethyl)sulfonyl]oxy}-2-naphthoate (221 mg, 0.449 mmol) from Example 1, Step 2, followed by 2M sodium carbonate (0.785 mL, 1.571 mmol) was added. The mixture was degassed again, PdCl₂(dppf).CH₂Cl₂ (17 mg, 0.023 mmol) was added and the mixture stirred at 85° C. for 2 h. It was cooled to room temperature, diluted with Et₂O and water and the solid was filtered off. The aqueous phase was extracted with EtOAc and the combined organic fractions were washed three times with water and then brine, dried (MgSO₄), filtered and the solvent was evaporated under reduced pressure. The residue was subjected to chromatography on silica gel (40 g), eluting with EtOAc/hexanes (0-50%) to give ethyl 4-[5-(2,2-difluoro-1-hydroxyethyl)-3-thienyl]-7-[4-(trifluoromethyl)phenyl]-2-naphthoate as a colorless solid. MS: M+H (+ESI)=507.2.

### Step 4

A solution of 4M lithium hydroxide (296 μL, 1.185 mmol) was added to a stirred mixture of ethyl 4-[5-(2,2-difluoro-1-hydroxyethyl)-3-thienyl]-7-[4-(trifluoromethyl)phenyl]-2-naphthoate (150 mg, 0.296 mmol) in methanol:THF (1:1, 0.6 mL) and the mixture was stirred at room temperature for 4 h. The mixture was diluted with EtOAc, hydrochloric acid (1 M, 3 mL) was added and the mixture was extracted twice with ethyl acetate (2 mL). The combined organic fractions were washed with saturated brine solution (3 mL), dried (MgSO₄), filtered and the solvent was evaporated under reduced pressure. The residue was purified by column chromatography on silica gel (12 g), eluting with EtOAc/hexanes (0-50%) containing 1% AcOH to afford 4-[5-(2,2-difluoro-1-hydroxyethyl)-3-thienyl]-7-[4-(trifluoromethyl)phenyl]-2-naphthoic acid as a colorless solid. MS: M−H (−ESI)=476.9.

### Example 10

### 4-(3-Thienyl)-7-[4-(trifluoromethoxy)phenyl]-2-naphthoic acid

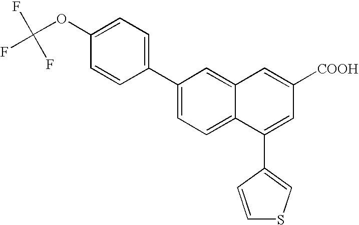

### Step 1

A suspension of ethyl 7-[4-(trifluoromethoxy)phenyl]-4-{[(trifluoromethyl)sulfonyl]oxy}-2-naphthoate (300 mg, 0.590 mmol), 3-thiopheneboronic acid (91 mg, 0.708 mmol) and 2M sodium carbonate (885 μL, 1.770 mmol) in DMF (4 mL) was degassed and PdCl₂(dppf).CH₂Cl₂ (22.36 mg, 0.031 mmol) was added. The mixture was heated to 80° C. and stirred for 3 h. It was cooled to room temperature, diluted with Et₂O and water and the solid was filtered off. The aqueous phase was extracted with EtOAc and the combined organic fractions were washed three times with water and then brine, dried (MgSO₄), filtered and the solvent was evaporated under reduced pressure. The residue was subjected to chromatography on silica gel (40 g), eluting with EtOAc/hexanes (0-45%) to give ethyl 4-(3-thienyl)-7-[4-(trifluoromethoxy)phenyl]-2-naphthoate as a yellowish solid.

MS: M+H (+ESI)=443.0.

### Step 2

The hydrolysis was carried out as described in Example 9, Step 4 to give 4-(3-thienyl)-7-[4-(trifluoromethoxy)phenyl]-2-naphthoic acid. MS: M−H (−ESI)=412.9.

### Example 11

### 3-Fluoro-4-(3-thienyl)-7-[4-(trifluoromethoxy)phenyl]-2-naphthoic acid

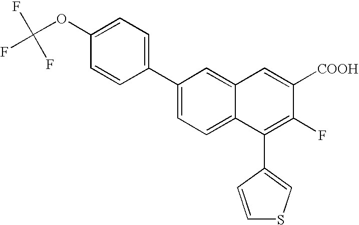

### Step 1

N-fluoropyridinium triflate (3.53 g, 14.27 mmol) was added to a stirred mixture of ethyl 7-bromo-4-hydroxy-2-naphthoate (3.51 g, 11.89 mmol) in chlorobenzene (60 mL) and the mixture was stirred at reflux temperature overnight. The mixture was cooled, diluted with EtOAc, washed with HCl, aqueous sodium bicarbonate, brine, dried (MgSO₄), filtered and the solvent was evaporated under reduced pressure. The residue was passed twice through a column of silica gel, eluting with EtOAc/hexanes (20-40%) to give ethyl 7-bromo-3-fluoro-4-hydroxy-2-naphthoate as an orange solid. MS: M−H (−ESI)=310.9, 312.9.

### Step 2

A suspension of ethyl 7-bromo-3-fluoro-4-hydroxy-2-naphthoate (230 mg, 0.720 mmol), 4-(trifluoromethoxy)benzene boronic acid, 2M Na₂CO₃ (1.08 mL, 2.16 mmol) and DMF (3.6 mL) was degassed and PdCl₂(dppf)-CH₂Cl₂ (29.4 mg, 0.036 mmol) was added. The mixture was heated at 80° C. for 45 min. It was cooled, diluted with ethyl ether, poured in water and extracted twice with ethyl ether. The organic layer was washed with water twice and then with brine, dried (MgSO₄), filtered and the solvent was evaporated under reduced pressure. The residue was purified by column chromatography on silica gel CombiFlash Silicycle 40 g, eluting with EtOAc/hexanes (0-60%) to give ethyl 3-fluoro-4-hydroxy-7-[4-(trifluoromethoxy)phenyl]-2-naphthoate as a yellow solid. MS: M+H (+ESI)=395.1); M−H (−ESI)=393.1.

### Step 3

Triflic anhydride (100 μL, 0.589 mmol) was added to a stirred, cooled (0° C.) mixture of ethyl 3-fluoro-4-hydroxy-7-[4-(trifluoromethoxy)phenyl]-2-naphthoate (202 mg, 0.512 mmol) and pyridine (62.1 μl, 0.768 mmol) in dichloromethane and the mixture was stirred at 0° C. for 2 h. The mixture was cooled, diluted with dichloromethane (20 mL), washed with brine, dried (MgSO₄), filtered and the solvent was evaporated under reduced pressure. The residue was purified by column chromatography on silica gel CombiFlash Silicycle 40 g, eluting with EtOAc/hexanes (0-25%) to give ethyl 3-fluoro-7-[4-(trifluoromethoxy)phenyl]-4-{[(trifluoromethyl)sulfonyl]oxy}-2-naphthoate as a colorless solid.

¹H NMR (500 MHz, acetone-d₆): δ 8.88 (m, 1H), 8.66 (s, 1H), 8.35 (m, 1H), 8.22 (m, 1H), 8.06 (m, 2H), 7.56 (m, 2H), 4.51 (m, 2H), 1.46 (m, 3H) ppm.

### Step 4

3-Fluoro-4-(3-thienyl)-7-[4-(trifluoromethoxy)phenyl]-2-naphthoic acid was prepared by using the same reaction sequence as for Example 10, Step 1 but using the triflate from the previous Step 3, followed by hydrolysis as in Example 9, Step 4. MS: M−H (−ESI)=489.0.

### Example 12

### 7-[(2,6-Dimethylbenzyl)oxy]-4-[(2-methoxybenzyl)oxy]-2-naphthoic acid

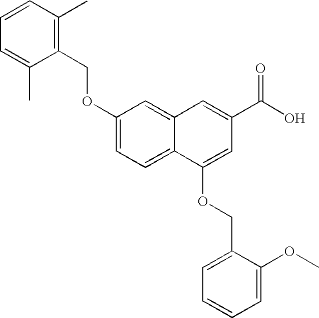

### Step 1

A mixture containing ethyl 7-(benzyloxy)-4-hydroxy-2-naphthoate (2.0 g, 6.2 mmol), 2-(trimethylsilyl)ethoxymethyl chloride (SEM chloride) (1.21 mL) and K₂CO₃ (1.52 g) in acetonitrile (50 mL) was heated to 55° C. for 1.5 h. EtOAc was then added and the solids were removed by filtration. The filtrate was concentrated and the residue subjected to flash chromatography on silica gel eluting with 2.5% EtOAc/toluene to give ethyl 7-(benzyloxy)-4-{[2-(trimethylsilyl)ethoxy]methoxy}-2-naphthoate as a yellowish oil.

### Step 2

The product of step 1 (9.3 g, 20.6 mmol) was dissolved in ethanol (90 mL) and EtOAc (45 mL). 10% Pd/C (930 mg) was then added and the mixture was stirred overnight under an atmosphere of hydrogen. The catalyst was removed by filtration through celite. Evaporation of the filtrate gave ethyl 7-hydroxy-4-{[2-(trimethylsilyl)ethoxy]methoxy}-2-naphthoate which was used in the next step without further purification.

### Step 3

A mixture containing the product of step 2 (7.33 g, 19.3 mmol), 2,6-dimethylbenzyl chloride (3.28 g), K₂CO₃ (3.2 g) and Bu₄NI (0.7 g) in acetonitrile (100 mL) was heated to 55° C. for 2 h. EtOAc was then added and the solids were removed by filtration. The filtrate was concentrated and the residue redissolved in EtOAc, washed with aqueous NH₄Cl and dried over Na₂SO₄. Purification by flash chromatography on silica gel eluting with toluene afforded the dimethylbenzyl ether as a yellowish oil.

### Step 4

A solution of the product of step 3 (7.82 g, 16.3 mmol) and carbon tetrabromide (1.35 g) in 2-propanol was heated to reflux temperature for 3 h. The solvent was then evaporated and the residue triturated with toluene (50 mL) to afford ethyl 7-[(2,6-dimethylbenzyl)oxy]-4-hydroxy-2-naphthoate as a white solid.

### Step 5

A solution of the product of step 4 (0.12 g, 0.34 mmol), 1-(chloromethyl)-2-methoxybenzene (0.059 g, 0.38 mmol), tetrabutylammonium iodide (0.013 mg, 0.034 mmol) and potassium carbonate (0.052 g, 0.38 mmol) in acetone was heated at reflux temperature for 5 h. The mixture was cooled to rt and diluted with EA and washed with aqueous NH₄Cl. The organic phase was dried over MgSO₄, filtered and the solvent removed. Chromatography on silica gel eluting with hexane:toluene (4:1) gave ethyl 7-[(2,6-dimethylbenzyl)oxy]-4-[(2-methoxybenzyl)oxy]-2-naphthoate.

### Step 6

2N Sodium hydroxide (0.187 mL, 0.37 mmol) was added to a solution of the product of Step 5 (88 mg) in THF (1 mL) and MeOH (1 mL). The reaction mixture was heated at 55° C. for 5 h. After cooling to rt, the solution was diluted with EA and quenched with 10% HCl. The organic phase was separated, dried over MgSO₄ and the solvent removed by evaporation under diminished pressure. The crude product was triturated with 2:1 hexanes/Et₂O to afford 7-[(2,6-dimethylbenzyl)oxy]-4-[(2-methoxybenzyl)oxy]-2-naphthoic acid. MS: M−H (−ESI)=441.3.

### Example 13

### 7-[(2,6-Dimethylbenzyl)oxy]-4-(3-thienyl)-2-naphthoic acid

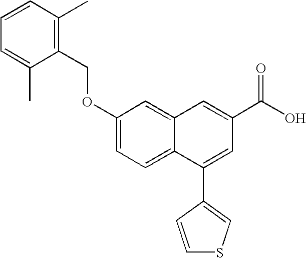

### Step 1

The intermediate from Example 12, Step 4, ethyl 7-[(2,6-dimethylbenzyl)oxy]-4-hydroxy-2-naphthoate, was treated with Tf₂O and pyridine as in Example 1, Step 2, to afford ethyl 7-[(2,6-dimethylbenzyl)oxy]-4-{[(trifluoromethyl)sulfonyl]oxy}-2-naphthoate. ¹H NMR (500 MHz, acetone-d₆): δ 8.7 (1H, s), 8.1 (1H, d), 7.95 (2H, d), 7.6 (1H, s), 7.1-7.3 (3H, m), 5.4 (2H, s), 4.45-4.55 (2H, q), 2.45 (6H, s), 1.4-1.5 (3H, t) ppm.

### Step 2

The title compound was prepared using the method described in Example 10, Steps 1 and 2, but using thiophene-3-boronic acid to afford 7-[(2,6-dimethylbenzyl)oxy]-4-(3-thienyl)-2-naphthoic acid. MS: M−H (−ESI)=386.8.

### Example 14

### 7-[(2,6-Dimethylbenzyl)oxy]-4-(4-formylphenyl)-2-naphthoic acid

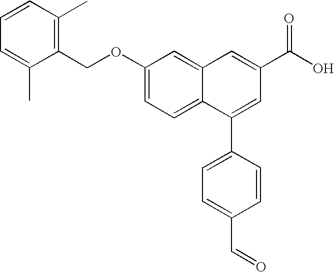

To a screw top test tube equipped with a magnetic stir bar was added 4-(formyl)benzeneboronic acid (7.5 mg, 0.050 mmol). Then a solution of dimethoxyethane (2 mL) which contained ethyl 7-[(2,6-dimethylbenzyl)oxy]-4-{[(trifluoromethyl)sulfonyl]oxy}-2-naphthoate (20 mg, 0.041 mmol) was added, followed by sodium carbonate (62 μL of a 2.0 M solution, 0.124 mmol). Nitrogen was bubbled through the solution with stirring and then Pd(PPh₃)₄ (4.8 mg, 0.004 mmol) was added. The test tube was purged with nitrogen and sealed with a cap. The reaction was heated for 16 h to 90° C. After cooling, it was filtered through a small plug of silica gel using a mixture of 10:1 acetonitrile:Et₃N and then the filtrate was evaporated. A stir bar was placed in the test tube from the above mixture and 2.3 mL (0.65 mmol of LiOH:H₂O) of a solution prepared as follows was added: to a 125 mL erlenmeyer flask was added (1.12 g, 26.7 mmol) of LiOH:H₂O followed by 39 mL of tetrahydrofuran, 29 mL, of methanol and 29 mL of water under stirring until LiOH completely dissolved. The reaction was stirred for 24 h at room temperature. It was quenched with 1 mL (1.3 mmol of formic acid) of a solution prepared as follows: in a separate 125 erlenmeyer flask, 2 mL (52.1 mmol) of formic acid was added to 40 mL of THF. The reaction was stirred for 10 min and the solvent was evaporated using centrifugal evaporation. The crude reaction mixture was dissolved in 1 mL of DMSO and purified using mass-directed preparative LC/MS. The product, 7-[(2,6-dimethylbenzyl)oxy]-4-(4-formylphenyl)-2-naphthoic acid, was isolated as an off-white solid.

MS: M−H (−ESI)=409.27.

### Example 15

### 7-[(2,6-Dimethylbenzyl)oxy]-4-(4-hydroxyphenyl)-2-naphthoic acid

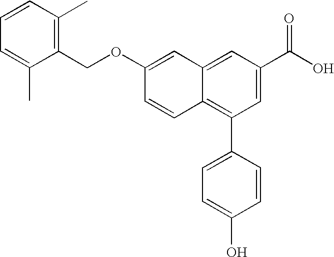

The title compound was prepared using the method described in Example 14 but using 4-hydroxybenzeneboronic acid (6.9 mg, 0.050 mmol) to afford 7-[(2,6-dimethylbenzyl)oxy]-4-(4-hydroxyphenyl)-2-naphthoic acid. MS: M−H (−ESI)=397.22.

### Example 16

### 4-(4-Carboxyphenyl)-7-[(2,6-dimethylbenzyl)oxy]-2-naphthoic acid

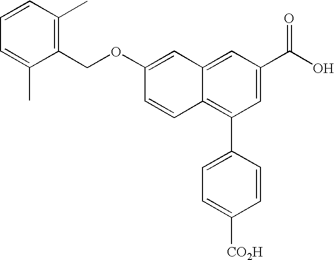

The title compound was prepared using the method described in Example 14 but using [4-(methoxycarbonyl)benzene]boronic acid (9.0 mg, 0.050 mmol) and subsequent saponification of the resulting methyl ester to afford 4-(4-carboxyphenyl)-7-[(2,6-dimethylbenzyl)oxy]-2-naphthoic acid. MS: M−H (−ESI)=425.26.

### Example 17

### 7-[(2,6-Dimethylbenzyl)oxy]-4-[4-(hydroxymethyl)phenyl]-2-naphthoic acid

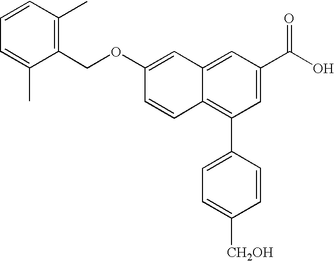

The title compound was prepared using the method described in Example 14 but using [4-(hydroxymethyl)benzene]boronic acid (7.6 mg, 0.050 mmol) to afford 7-[(2,6-dimethylbenzyl)oxy]-4-[4-(hydroxymethyl)phenyl]-2-naphthoic acid. MS: M−H (−ESI)=411.26.

### Example 18

### 4-(1,3-Benzodioxol-5-yl)-7-[(2,6-dimethylbenzyl)oxy]-2-naphthoic acid

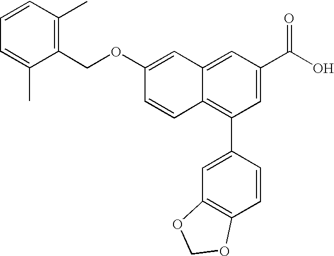

The title compound was prepared using the method described in Example 14 but using (1,3-benzodioxol-5-yl)boronic acid (8.3 mg, 0.050 mmol) to afford 4-(1,3-benzodioxol-5-yl)-7-[(2,6-dimethylbenzyl)oxy]-2-naphthoic acid. MS: M−H (−ESI)=425.23.

### Example 19

### 7-{[2-Methyl-5-(trifluoromethyl)benzyl]oxy}-4-(3-thienyl)-2-naphthoic acid

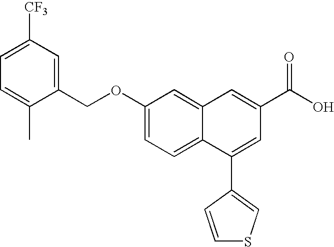

To a screw-top test tube fitted with a stir bar was added 2-methyl-5-(trifluoromethyl)benzyl chloride (42.0 mg, 0.201 mmol) followed by 1 mL of a triglyme solution of ethyl 7-hydroxy-4-(3-thienyl)-2-naphthoate (40 mg, 0.134 mmol). Then potassium carbonate (37.1 mg, 0.268 mmol) was added and the reaction set to stir after bubbling nitrogen through the reaction tube. The reaction was stirred over 48 h. To the test tube was added 51 μL of tetraethylenepentamine (50.7 mg, 0.268 mmol), to react with any excess benzyl chloride, and the reaction allowed to stir for 3 h. Then 4.6 mL (1.3 mmol of LiOH:H₂O) of the following solution was added: to a 125 mL erlenmeyer flask was added (1.12 g, 26.7 mmol) of LiOH:H₂O, and then 39 mL of tetrahydrofuran, 29 mL of methanol and 29 mL of water. The mixture was stirred until all the LiOH dissolved. The reaction was stirred for 24 h at room temperature and then the reaction was quenched with 2 mL (2.6 mmol of formic acid) of the following solution: in a separate 125 erlenmeyer flask was added 2 mL (52.1 mmol) of formic acid to 40 mL of THF. The mixture was stirred reaction for 10 min and concentrated using centrifugal evaporation. The crude reaction mixture was diluted with 1 mL of DMSO and purified using mass-directed preparative LC/MS. The product, 7-{[2-methyl-5-(trifluoromethyl)benzyl]oxy}-4-(3-thienyl)-2-naphthoic acid, was obtained as an off-white solid. MS: M−H (−ESI)=441.19.

### Example 20

### 7-[(2,6-Dichlorobenzyl)oxy]-4-(3-thienyl)-2-naphthoic acid

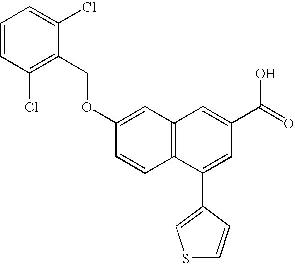

The title compound was prepared using the method described in Example 19 but using 2,6-dichlorobenzyl bromide (48.3 mg, 0.201 mmol) to afford 7-[(2,6-dichlorobenzyl)oxy]-4-(3-thienyl)-2-naphthoic acid. MS: M−H (−ESI)=427.08.

### Example 21

### 7-[(3,5-Dichloropyridin-4-yl)methoxy]-4-(3-thienyl)-2-naphthoic acid

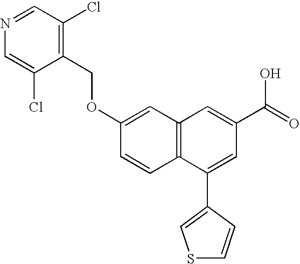

### Step 1: 4-(Bromomethyl)-3,5-dichloropyridine

To a suspension of (3,5-dichloropyridin-4-yl)methanol (1.3 g, 7.3 mmol), triphenylphosphine (2.30 g, 8.76 mmol), imidazole (600 mg, 8.76 mmol) in 18 mL of solvent (7 mL acetonitrile and 11 mL diethyl ether) at 0° C., was added bromine (450 μL, 8.76 mmol) and the solution was allowed to stir at 0° C. for 30 min. The reaction was then quenched with sodium metabisulfite and the aqueous layer was washed three times with ether (50 mL). The combined organic layers were then washed with brine, dried over sodium sulfate and the solvent evaporated to afford a crude oil which crystallized upon cooling to give 4-(bromomethyl)-3,5-dichloropyridine as a yellow solid. ¹H NMR (500 MHz, acetone-d₆): δ 8.60 (s, 2H), 4.78 (s, 2H) ppm.

### Step 2

The title compound was prepared using the method described in Example 19 but using 4-(bromomethyl)-3,5-dichloropyridine (48.5 mg, 0.201 mmol) to give 7-[(3,5-dichloropyridin-4-yl)methoxy]-4-(3-thienyl)-2-naphthoic acid. MS: M−H (−ESI)=428.10.

### Example 22

### 7-{[2-Chloro-5-(trifluoromethyl)benzyl]oxy}-4-(3-thienyl)-2-naphthoic acid

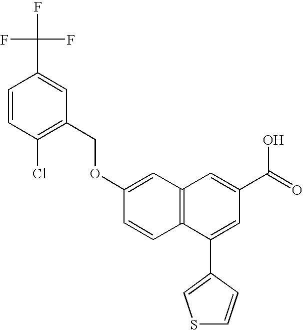

The title compound was prepared using the method described in Example 19 but using 2-chloro-5-(trifluoromethyl)benzyl bromide (55.0 mg, 0.201 mmol) to afford 7-{[2-chloro-5-(trifluoromethyl)benzyl]oxy}-4-(3-thienyl)-2-naphthoic acid. MS: M−H (−ESI)=461.11.

### Example 23

### 7-[(2-Chloro-6-fluorobenzyl)oxy]-4-(3-thienyl)-2-naphthoic acid

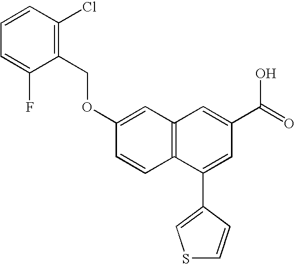

The title compound was prepared using the method described in Example 19 but using 2-chloro-6-fluorobenzyl bromide (44.9 mg, 0.201 mmol) to afford 7-[2-chloro-6-fluorobenzyl)oxy]-4-(3-thienyl)-2-naphthoic acid. MS: M−H (−ESI)=411.12.

### Example 24

### 4-(3-Thienyl)-7-[(2,4,6-trifluorobenzyl)oxy]-2-naphthoic acid

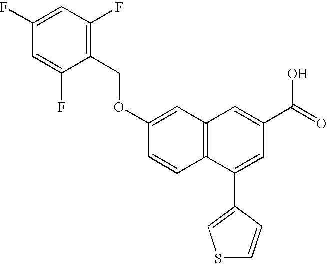

The title compound was prepared using the method described in Example 19 but using 2,4,6-trifluorobenzyl bromide (45.3 mg, 0.201 mmol) to afford 4-(3-thienyl)-7-[(2,4,6-trifluorobenzyl)oxy]-2-naphthoic acid. MS: M−H (−ESI)=413.15.

### Example 25

### 7-[(2,6-Difluorobenzyl)oxy]-4-(3-thienyl)-2-naphthoic acid

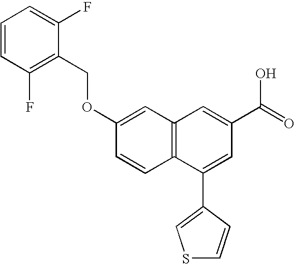

The title compound was prepared using the method described in Example 19 but using 2,6-difluorobenzyl bromide (41.6 mg, 0.201 mmol) to afford 7-[(2,6-difluorobenzyl)oxy]-4-(3-thienyl)-2-naphthoic acid. MS: M−H (−ESI)=395.16.

### Example 26

### 7-[(6-Chloro-2-fluoro-3-methylbenzyl)oxy]-4-(3-thienyl)-2-naphthoic acid

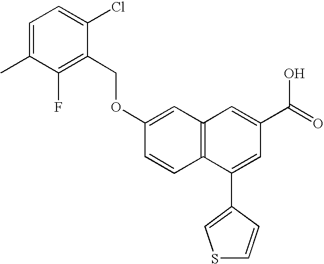

The title compound was prepared using the method described in Example 19 but using 6-chloro-2-fluoro-3-methylbenzyl bromide (47.8 mg, 0.201 mmol) to afford 7-[(6-chloro-2-fluoro-3-methylbenzyl)oxy]-4-(3-thienyl)-2-naphthoic acid. MS: M−H (−ESI)=425.16.

### Example 27

### 7-[(2-Bromo-6-chlorobenzyl)oxy]-4-(3-thienyl)-2-naphthoic acid

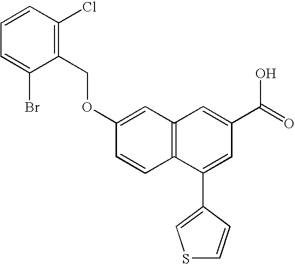

The title compound was prepared using the method described in Example 19 but using 2-bromo-6-chlorobenzyl bromide (57.2 mg, 0.201 mmol) to afford 7-[(2-bromo-6-chlorobenzyl)oxy]-4-(3-thienyl)-2-naphthoic acid. MS: M−H (−ESI)=473.05.

### Example 28

### 7-[(2,6-Dibromobenzyl)oxy]-4-(3-thienyl)-2-naphthoic acid

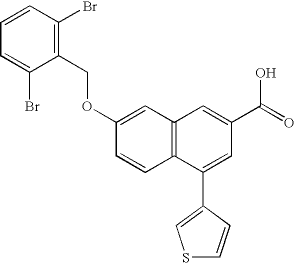

### Step 1: 2,6-Dibromobenzyl bromide

This compound was prepared according to the procedure described by A. van den Hoogenband, et al., in Tetrahedron Lett., 47: 4361-4364 (2006).

### Step 2

The title compound was prepared using the method described in Example 19 but using 2,6-dibromobenzyl bromide (66.1 mg, 0.201 mmol) to afford 7-[(2,6-dibromobenzyl)oxy]-4-(3-thienyl)-2-naphthoic acid. MS: M−H (−ESI)=517.01.

### Example 29

### 4-(Benzyloxy)-7-[(2,6-dimethylbenzyl)oxy]-2-naphthoic acid

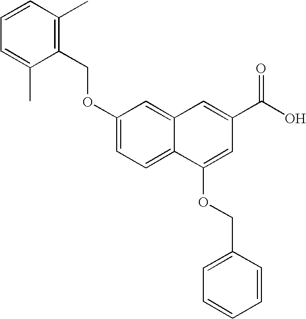

The title compound was prepared using the method described in Example 12 but using benzyl bromide to afford 4-(benzyloxy)-7-[(2,6-dimethylbenzyl)oxy]-2-naphthoic acid.

MS: M−H (−ESI)=411.0.

### Example 30

### 4-(4-Acetylphenyl)-7-[(2,6-dimethylbenzyl)oxy]-2-naphthoic acid

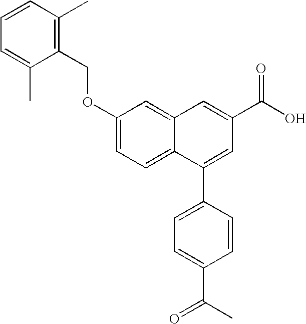

The title compound was prepared following the procedure of Example 13 but using 4-acetylbenzeneboronic acid in place of thiophene-3-boronic acid to afford 4-(4-acetylphenyl)-7-[(2,6-dimethylbenzyl)oxy]-2-naphthoic acid. MS: M−H (−ESI)=423.0.

### Example 31

### 7-[(2,6-Dimethylbenzyl)oxy]-4-[4-(methylsulfonyl)phenyl]-2-naphthoic acid

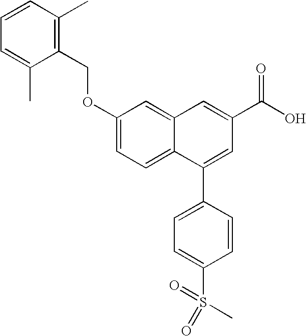

The title compound was prepared following the procedure of Example 13 but using 4-(methylsulfonyl)benzeneboronic acid in place of thiophene-3-boronic acid to afford 7-[(2,6-dimethylbenzyl)oxy]-4-[4-(methylsulfonyl)phenyl]-2-naphthoic acid.

MS: M−H (−ESI)=459.0.

### Example 32

### 7-[2,6-Dimethylbenzyl)oxy]-4-[4-(1-hydroxy-1-methylethyl)phenyl]-2-naphthoic acid

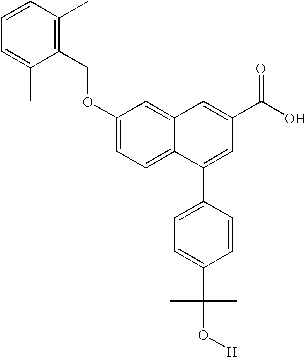

### Step 1

To a suspension of 4-acetylbenzeneboronic acid (5 g, 5 mmol) in diethyl ether at 0° C. was added 2M methylmagnesium chloride (60 mL, 180 mmol). The mixture was stirred at RT for 30 min and quenched with the addition of 3N HCl. The product was extracted with EA and the organic layer was dried as usual. After removal of the solvent, the residue was subjected to chromatography on SiO₂ using acetone:toluene:acetic acid (30:70:1) as eluant to give the boronic acid used in Step 2 without further purification. ¹H NMR (500 MHz, acetone-d₆): δ 7.8 (2H, d), 7.5 (2H, d), 1.5 (6H, s) ppm.

### Step 2

The title compound was prepared following the procedure of Example 30 but using the benzeneboronic acid from Step 1 in place of thiophene-3-boronic acid to afford 7-[(2,6-dimethylbenzyl)oxy]-4-[4-(1-hydroxy-1-methylethyl)phenyl]-2-naphthoic acid.

MS: M−H (−ESI)=439.0.

### Example 33

### 7-[(4-Fluoro-2,6-dimethylbenzyl)oxy]-4-(3-thienyl)-2-naphthoic acid

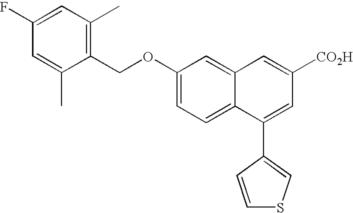

### Step 1

To diisopropylamine (16.23 mL, 114 mmol) in THF (236 mL) at 0° C. was added nBuLi (44.6 mL, 112 mmol). The mixture was stirred for 15 min at 0° C. A solution of 4-tert-butyl 1-ethyl 2-(diethoxyphosphoryl)succinate (38.9 g, 115 mmol) in THF (3 mL) was then added, stirred for 15 min at 0° C., and then a solution of 3-(benzyloxy)benzaldehyde (23.69 g, 112 mmol) in THF (3 mL) was added. The mixture was allowed to warm to room temperature and stirred overnight. It was diluted with brine, extracted with EtOAc, washed with brine, dried with Na₂SO₄, filtered and evaporated. The residue was subjected to column chromatography on silica gel, eluting with 8% EtOAc/hexane to give 4-tert-butyl 1-ethyl (2E)-2-[3-(benzyloxy)benzylidene]succinate as a colorless oil.

### Step 2

To 4-tert-butyl 1-ethyl (2E)-2-[3-(benzyloxy)benzylidene]succinate (22.6 g, 57.0 mmol) in dichloromethane (452 mL) at 0° C. was added trifluoroacetic acid (110 mL, 1425 mmol). The mixture was stirred 6 h at 0° C. The solvent was evaporated under vacuum at 0° C. and co-evaporated twice with hexane to give (3E)-4-[3-(benzyloxy)phenyl]-3-(ethoxycarbonyl)but-3-enoic acid.

### Step 3

To (3E)-4-[3-(benzyloxy)phenyl]-3-(ethoxycarbonyl)but-3-enoic acid (82 g, 241 mmol) was added acetic anhydride (1268 mL) and sodium acetate (19.84 g, 242 mmol). The mixture was heated at 70° C. for 30 min. The solvent was evaporated under vacuum. Ethanol (1268 mL) and potassium carbonate (66.6 g, 482 mmol) were added. The mixture was heated at 70° C. for 3 h and then at 80° C. for 4 h. It was acidified with 1N HCl and extracted three times with ether. The combined organic extracts were washed with brine, dried with Na₂SO₄, filtered, evaporated and co-evaporated with toluene. Purification was achieved by flash silica gel chromatography to give ethyl 7-(benzyloxy)-4-hydroxy-2-naphthoate.

### Step 4

To a suspension of ethyl 7-(benzyloxy)-4-hydroxy-2-naphthoate (15 g, 46.5 mmol) in CH₂Cl₂ (212 mL) in an ice/water bath, was added pyridine (5.65 mL, 69.8 mmol) and Tf₂O (9.59 mL, 56.8 mmol). The mixture was stirred at ice temperature for 1 h. Saturated NH₄Cl was added and the mixture was extracted with EtOAc. It was dried with Na₂SO₄, filtered and evaporated to give ethyl 7-(benzyloxy)-4-{[(trifluoromethyl)sulfonyl]oxy}-2-naphthoate.

### Step 5

To ethyl 7-(benzyloxy)-4-{[(trifluoromethyl)sulfonyl]oxy}-2-naphthoate (21.13 g, 46.5 mmol) was added dioxane (224 mL), water (9 mL), thiophene-3-boronic acid (7.26 g, 56.7 mmol), 2-(dicyclohexylphosphino)biphenyl (0.978 g, 2.79 mmol), tripotassium phosphate (12.24 g, 57.7 mmol) and palladium(II) acetate (0.522 g, 2.325 mmol). The mixture was degassed (N₂ bubbling) for 10 min and then heated at 80° C. for 1 h. It was cooled to RT and extracted with EtOAc. The EA layer was washed twice with saturated NH₄Cl, dried with Na₂SO₄ and filtered. The solution was filtered through a small pad of silica gel eluting with EtOAc. The solvent was evaporated and the residue dissolved in DMSO (10 mL), THF (40 mL) and toluene. It was filtered on silica gel using toluene as the mobile phase and, after evaporation of the solvent, the residue was triturated overnight with ether. The solid was filtered and dried to give ethyl 7-(benzyloxy)-4-(3-thienyl)-2-naphthoate as a gray solid.

### Step 6

To ethyl 7-(benzyloxy)-4-(3-thienyl)-2-naphthoate (2 g, 5.15 mmol) in CH₂Cl₂ (51.5 mL) in an ice/water bath was added boron tribromide as a 1M CH₂Cl₂ solution (7.72 mL, 7.72 mmol). The mixture was stirred 45 min in the ice bath and then poured onto ice/saturated NaHCO₃. It was extracted with EtOAc, washed with brine, dried with Na₂SO₄, filtered and evaporated. After evaporation of the solvent, the residue was triturated overnight with ether to give ethyl 7-hydroxy-4-(3-thienyl)-2-naphthoate.

### Step 7

To a stirred solution of ethyl 7-hydroxy-4-(3-thienyl)-2-naphthoate (150 mg, 0.503 mmol) and 2,6-dimethyl-4-fluorobenzyl bromide (142 mg, 0.654 mmol) in acetonitrile (15 mL) at room temperature was added potassium carbonate (104 mg, 0.754 mmol) in one portion. The resulting mixture was stirred at 60° C. overnight, diluted with ethyl acetate, filtered through a silica gel pad, and evaporated. The residue was re-dissolved in a mixture of 10 mL THF and 10 mL of MeOH and the solution was treated with 5 mL of 2 N NaOH at 50° C. for 2 h. The reaction was worked up by the addition of hydrochloric acid followed by extraction with ethyl acetate, drying over Na₂SO₄, and evaporation. The residue was triturated with diethyl ether and the solid filtered and dried to afford the desired 7-[(4-fluoro-2,6-dimethylbenzyl)oxy]-4-(3-thienyl)-2-naphthoic acid as a white solid. MS: M−H (−ESI)=405.0.

### Example 34

### 4-Phenyl-7-[(2,3,6-trichlorobenzyl)oxy]-2-naphthoic acid

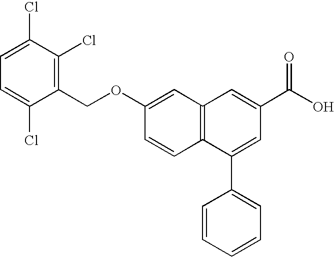

To a stirred solution of methyl 7-hydroxy-4-phenyl-2-naphthoate (400 mg, 1.437 mmol; J. Med. Chem. 39, 1996, 3951) and 2,3,6-trichlorobenzyl bromide (473 mg, 1.725 mmol) in acetonitrile (25 mL) at room temperature was added potassium carbonate (238 mg, 1.725 mmol) in one portion. The mixture was stirred at 70° C. for 4 h. The reaction was worked up by filtration and evaporation. The residue was then dissolved in 15 mL of MeOH and 15 mL of THF and the solution was treated with 2 mL of NaOH (10 N) at 60° C. for 2 h. It was acidified with 10 mL of 2.2 MH₃PO₄ and extracted with EtOAc. The organic layer was dried as usual and evaporated. After removal of the solvent, the residue was triturated with ether, the solid filtered and dried to afford 4-phenyl-7-[(2,3,6-trichlorobenzyl)oxy]-2-naphthoic acid as a white solid.

MS: M−H (−ESI)=455.0.

### Example 35

### 4-(3-Thienyl)-7-[(2,3,6-trichlorobenzyl)oxy]-2-naphthoic acid

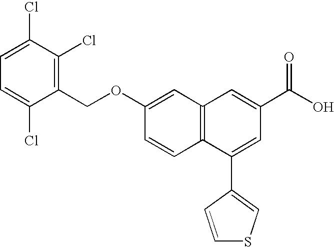

To a stirred solution of ethyl 7-hydroxy-4-(3-thienyl)-2-naphthoate (150 mg, 0.503 mmol) and 2,3,6-trichlorobenzyl bromide (179 mg, 0.654 mmol) in acetonitrile (15 mL) at room temperature was added potassium carbonate (104 mg, 0.754 mmol) in one portion. The resulting mixture was stirred at 60° C. overnight. It was diluted with ethyl acetate, filtered through a silica gel pad, and evaporated. After removal of the solvent, the residue was re-dissolved in a mixture of 10 mL THF and 10 mL of MeOH and the solution treated with 5 mL of 2 N of NaOH at 50° C. for 2 h. The reaction was worked up by the addition of hydrochloric acid, extracted with ethyl acetate, dried over Na₂SO₄, and evaporated. The residue was triturated with diethyl ether and the resulting solid filtered and dried to afford the desired 4-(3-thienyl)-7-[(2,3,6-trichlorobenzyl)oxy]-2-naphthoic acid as a white solid. MS: M−H (−ESI)=462.8.

### Example 36

### 4-(4-Imidazo[1,2-a]pyrimidin-2-yl-phenyl)-7-(4-trifluoromethylphenyl)-2-naphthoic acid

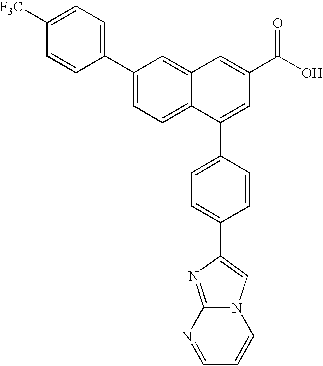

To a mixture of ethyl 4-(4,4,5,5-tetramethyl-1,3,2-dioxaborolan-2-yl)-7-[4-(trifluoromethyl)phenyl]-2-naphthoate (50 mg, 0.106 mmol) from Example 3, Step 1, PdCl₂(dppf)-CH₂Cl₂ adduct (8.68 mg, 10.63 μmol), 2-(4-bromo-phenyl)-imidazo[1,2-a]pyrimidine (43.7 mg, 0.159 mmol) under vacuum, was added DMF (2 ml) and 2 M potassium carbonate (0.159 mL, 0.319 mmol). The mixture was stirred under a nitrogen atmosphere at 90° C. for 3 h. The reaction was worked up by the addition of water, extracted with ethyl acetate, dried over Na₂SO₄, and evaporated. The solvent was evaporated and the residue was purified by Combiflash (0-100% EtOAc/hexane) chromatography to afford the desired ester. The ester was dissolved in 1 mL of THF and 1 mL of MeOH and treated with 1 mL of 2 N KOH at RT for 3 h. The reaction was worked up by the addition of aqueous citric acid, extracted with ethyl acetate, dried over Na₂SO₄, and evaporated. The residue was purified by Combiflash chromatography (0-30% solvent A/DCM with solvent A being a mixture of concentrated ammonia and MeOH (1:4)) to afford the desired 4-(4-imidazo[1,2-a]pyrimidin-2-yl-phenyl)-7-(4-trifluoromethyl-phenyl)-2-naphthoic acid as a solid. MS: M+H (+ESI)=510.0.

### Example 37

### 4-[4-(4H-[1,2,4]Triazol-3-yl)-phenyl]-7-(4-trifluoromethylphenyl)-2-naphthoic acid

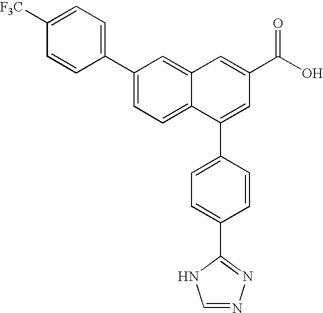

To a mixture of ethyl 4-(4,4,5,5-tetramethyl-1,3,2-dioxaborolan-2-yl)-7-[4-(trifluoromethyl)phenyl]-2-naphthoate (50 mg, 0.106 mmol) from Example 3, Step 1, PdCl₂(dppf)-CH₂Cl₂ adduct (8.68 mg, 10.63 μmol), 3-(4-bromophenyl)-1H-1,2,4-triazole (35.7 mg, 0.159 mmol) under vacuum, was added DMF (2 mL) and 2 M potassium carbonate (0.159 ml, 0.319 mmol). The mixture was stirred under a nitrogen atmosphere at 90° C. for 3 h. The reaction was worked up by the addition of water, extracted with ethyl acetate, dried over Na₂SO₄, and evaporated. The residue was purified by Combiflash chromatography (0-100% EtOAc/hexane) to afford the desired intermediate ester. The ester was dissolved in 2 mL of THF and 1 mL of MeOH and treated with 1 mL of 2 N KOH at rt for 3 h. The reaction was worked up by the addition of aqueous citric acid, extracted with ethyl acetate, dried over Na₂SO₄, and evaporated. The residue was purified by Combiflash chromatography (0-30% solvent A/DCM with solvent A being a mixture of concentrated ammonia and MeOH (1:4)) to afford the desired 4-[4-(4H-[1,2,4]triazol-3-yl)-phenyl]-7-(4-trifluoromethylphenyl)-2-naphthoic acid as a solid. MS: M+H (+ESI)=460.0 and M−H (−ESI)=458.0.

### Example 38

### 4-[4-(4-hydroxypiperidin-4-yl)phenyl]-7-[4-(trifluoromethyl)phenyl]-2-naphthoic acid

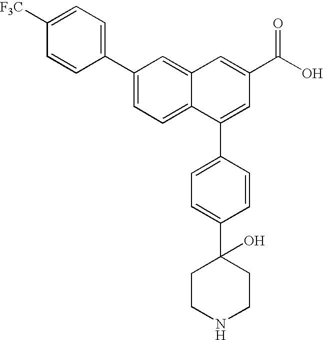

### Step 1

To a mixture of ethyl 4-(4,4,5,5-tetramethyl-1,3,2-dioxaborolan-2-yl)-7-[4-(trifluoromethyl)phenyl]-2-naphthoate (470 mg, 1 mmol) from Example 3, Step 1, PdCl₂(dppf)-CH₂Cl₂ adduct (82 mg, 0.1 mmol), 4-(4-bromophenyl)-4-piperidinol (384 mg, 1.5 mmol) under vacuum, was added DMF (10 mL) and 2 M potassium carbonate (1.5 mL, 3 mmol). The mixture was stirred under a nitrogen atmosphere at 95° C. for 3.5 h. The reaction was quenched by the addition of water. It was extracted with ethyl acetate and diethylether and the combined extracts were filtered on celite, dried over Na₂SO₄, and evaporated. The residue was purified by chromatography on SiO₂ using a mixture of concentrated aqueous NH₄OH, methanol and dichloromethane (1:9:90) to afford the desired intermediate ester.

¹H NMR (500 MHz, methanol-d₄): δ 8.71 (s, 1H), 8.40 (s, 1H), 8.04-7.94 (m, 4 μl), 7.91 (d, 1H), 7.81 (d, 2H), 7.72 (d, 2H), 7.53 (d, 2H), 4.46 (q, 2H), 3.38 (d, 2H), 3.20 (d, 2H), 2.26 (td, 2H), 1.96 (d, 2H), 1.46 (t, 3H).

### Step 2

The ester from Step 1 was dissolved in 7.2 mL of THF, 3.6 mL of MeOH and treated with 1.35 mL of 2 N LiOH at 55° C. for 3 h. Most of the solvent were removed under vacuum and dilute aqueous HCl was added carefully to a pH of about 5 yielding a suspension to which was added ethyl acetate, THF and brine. More dilute aqueous HCl was added to bring the mixture to a slightly more acidic pH. The mixture was extracted with EA. The combined extracts were dried with Na₂SO₄ and subjected to purification using reverse phase chromatography on a Phenomenex Max-RP column (100×21) and eluting with a gradient of 20% to 50% of acetonitrile in water containing 0.6% formic acid over 7.5 min at a flow rate of 25 mL/min. The product eluting at 4.6 min was collected and the solvent were removed in vacuo to yield the title compound.

¹H NMR (500 MHz, acetone-d₆): δ 8.65 (s, 1H), 8.54 (s, 1H), 8.37 (s, 1H), 8.08-8.02 (m, 3H), 7.94-7.83 (m, 4H), 7.66 (d, 2H), 7.52 (d, 2H), 3.25 (d, 4H), 2.37 (s, 2H), 1.81 (d, 2H).

MS: M+H (+ESI)=492.1.

### Example 39

### 4-(4-{3-Carboxy-6-[4-(trifluoromethyl)phenyl]-1-naphthyl}phenyl)piperidinium methanesulfonate

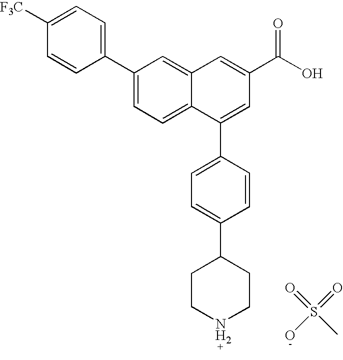

### Step 1

A mixture of ethyl 4-(4,4,5,5-tetramethyl-1,3,2-dioxaborolan-2-yl)-7-[4-(trifluoromethyl)phenyl]-2-naphthoate (6 g, 12.76 mmol) as prepared in Example 3, Step 1,4-(4-bromophenyl)piperidine (3.68 g, 15.31 mmol) and PdCl₂(dppf) (0.467 g, 0.638 mmol) in dioxane (42 mL) and 2M sodium carbonate (19.14 ml, 38.3 mmol) was degassed (vacuum-N₂ cycles). The mixture was heated to 85° C. and stirred for 2 h. The mixture was cooled down to RT, diluted with EtOAc and filtered. The aqueous phase was extracted with EtOAc and the combined organic fractions washed with brine, dried on MgSO₄, filtered and the solvent was evaporated under reduced pressure. The residue was purified by column chromatography on silica gel, eluting with MeOH/DCM (0-20%, spiked with 5% NEt₃) to give ethyl 4-(4-piperidin-4-ylphenyl)-7-[4-(trifluoromethyl)phenyl]-2-naphthoate as a brown solid. MS: M+H (+ESI)=504.2.

### Step 2

Methanesulfonic acid (0.493 mL, 7.59 mmol) was added to a stirred mixture of ethyl 4-(4-piperidin-4-ylphenyl)-7-[4-(trifluoromethyl)phenyl]-2-naphthoate (3.82 g, 7.59 mmol) in tetrahydrofuran (25 mL) and the mixture was stirred at 0° C. for 5 min. The precipitated solid was filtered, air-dried and triturated with EtOH/hexanes (80:20). The solid was collected by filtration and air-dried to give 4-(4-{3-(ethoxycarbonyl)-6-[4-(trifluoromethyl)phenyl]-1-naphthyl}phenyl)piperidinium methanesulfonate as a colorless solid. MS: M+H (+ESI)=504.2.

### Step 3

BOC₂O (2.55 g, 11.67 mmol) and triethylamine (1.627 mL, 11.67 mmol) were added to a stirred, cooled (0° C.) mixture of 4-(4-{3-(ethoxycarbonyl)-6-[4-(trifluoromethyl)phenyl]-1-naphthyl}phenyl)piperidinium methanesulfonate (2.8 g, 4.67 mmol) in methanol (8 mL) and the mixture was stirred at room temperature for 45 min. Silica gel was added and the volatiles were removed in vacuo. The residue was purified by column chromatography on silica gel, eluting with EtOAc/hexanes (0-50%) to give tent-butyl 4-(4-{3-(ethoxycarbonyl)-6-[4-(trifluoromethyl)phenyl]-1-naphthyl}phenyl)piperidine-1-carboxylate as a colorless solid. MS: M+Na (+ESI)=626.2.

### Step 4

A solution of 4M lithium hydroxide (8 mL, 32.0 mmol) was added to a stirred mixture of tert-butyl 4-(4-{3-(ethoxycarbonyl)-6-[4-(trifluoromethyl)phenyl]-1-naphthyl}phenyl)piperidine-1-carboxylate (2.76 g, 4.57 mmol) in THF:MeOH:DMSO (24 mL, 1:1:1) and the mixture was stirred at 80° C. for 18 h. HCl was added until acidic pH (<2) and the solution was extracted with EtOAc. The organic fractions were washed with brine, dried with MgSO₄, filtered and evaporated under reduced pressure. The residue was purified by column chromatography on silica gel, eluting with EtOAc/hexanes (0-10%, then spiked with 1% AcOH from 10-50%) to give 4-{4-[1-(tert-butoxycarbonyl)piperidin-4-yl]phenyl}-7-[4-(trifluoromethyl)phenyl]-2-naphthoic acid as a colorless solid. MS: M−H (−ESI)=574.2.

### Step 5

TFA (3.88 mL, 50.4 mmol) was added to a stirred mixture of 4-{4-[1-(tert-butoxycarbonyl)piperidin-4-yl]phenyl}-7-[4-(trifluoromethyl)phenyl]-2-naphthoic acid (2.9 g, 5.04 mmol) in dichloromethane and the mixture was stirred at room temperature for 90 min. The volatiles were then removed in vacuo and residual TFA was azeotroped with heptane and toluene. The residue (TFA salt) was suspended in DCM and methanesulfonic acid (MsOH) (0.35 mL, 5.39 mmol) was added. The first solid was dissolved, but another one quickly precipitated out (MsOH salt). This mixture was stirred an additional h, then the volatiles were removed in vacuo and TFA azeotroped with toluene. The solid was swished twice in dioxane/DCM 1/2 (30 mL), dissolved in water which was lyophilized to give 4-(4-{3-carboxy-6-[4-(trifluoromethyl)phenyl]-1-naphthyl}phenyl)piperidinium methanesulfonate as a colorless solid.

¹H NMR (400 MHz, methanol-d₄): δ 8.78 (s, 1H), 8.46 (s, 1H), 8.07-7.93 (m, 5H), 7.84 (d, 2H), 7.54 (q, 4H), 3.60 (d, 2H), 3.24 (t, 2H), 3.09 (t, 1H), 2.74 (s, 3H), 2.24 (d, 2H), 2.05 (q, 2H). MS: M+H (+ESI) 476.2; M−H (−ESI) 474.1.

### EXAMPLES OF PHARMACEUTICAL COMPOSITIONS

As a specific embodiment of an oral composition of a compound of the present invention, 50 mg of the compound of any of the Examples is formulated with sufficient finely divided lactose to provide a total amount of 580 to 590 mg to fill a size 0 hard gelatin capsule.

As a second specific embodiment of an oral composition of a compound of the present invention, 100 mg of the compound of any of the Examples, microcrystalline cellulose (124 mg), croscarmellose sodium (8 mg), and anhydrous unmilled dibasic calcium phosphate (124 mg) are thoroughly mixed in a blender; magnesium stearate (4 mg) and sodium stearyl fumarate (12 mg) are then added to the blender, mixed, and the mix transferred to a rotary tablet press for direct compression. The resulting tablets are optionally film-coated with Opadry® II for taste masking.

While the invention has been described and illustrated in reference to specific embodiments thereof, those skilled in the art will appreciate that various changes, modifications, and substitutions can be made therein without departing from the spirit and scope of the invention. For example, effective dosages other than the preferred doses as set forth hereinabove may be applicable as a consequence of variations in the responsiveness of the human being treated for a particular condition. Likewise, the pharmacologic response observed may vary according to and depending upon the particular active compound selected or whether there are present pharmaceutical carriers, as well as the type of formulation and mode of administration employed, and such expected variations or differences in the results are contemplated in accordance with the objects and practices of the present invention. It is intended therefore that the invention be limited only by the scope of the claims which follow and that such claims be interpreted as broadly as is reasonable.

1. A compound of structural formula I: 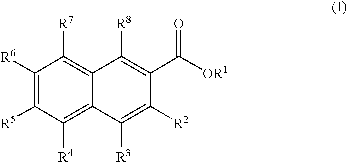
or a pharmaceutically acceptable salt thereof, wherein:
R¹ is selected from the group consisting of
hydrogen,
C₃₋₆ cycloalkyl,
benzyl, and
C₁₋₆ alkyl wherein alkyl is optionally substituted with hydroxy, amino, C₁₋₄ alkylamino, di-(C₁₋₄ alkyl)amino, aminocarbonyl, C₁₋₄ alkylaminocarbonyl, di-(C₁₋₄ alkyl)aminocarbonyl, C₁₋₄ alkylcarbonyloxy, C₁₋₄ alkyloxy, or one to five fluorines;
R² is hydrogen, fluorine, or hydroxy;
R³ is selected from the group consisting of:
—(CH₂)ₘaryl,
—(CH₂)ₘheteroaryl,
—OCH₂-aryl,
—OCH₂-heteroaryl,
—(S)ᵣCH₂-aryl,
—(S)ᵣCH₂-heteroaryl,
—CH₂O-aryl,
—CH₂O-heteroaryl,
—CH₂(S)ᵣ-aryl, and
—CH₂(S)ᵣ-heteroaryl;
wherein any methylene (CH₂) carbon atom in R³ is optionally substituted with one to two groups independently selected from fluorine, hydroxy, and C₁₋₄ alkyl optionally substituted with one to three fluorines; or two substituents when on the same methylene (CH₂) group are taken together with the carbon atom to which they are attached to form a cyclopropyl group; and wherein aryl and heteroaryl are optionally substituted with one to three R^{c} substituents independently selected from the group consisting of:
halogen,
cyano,
nitro,
C₁₋₆ alkoxy, wherein alkoxy is optionally substituted with one to five substituents independently selected from fluorine, hydroxy, and C₁₋₃ alkoxy,
C₁₋₆ alkyl, wherein alkyl is optionally substituted with one to five substituents independently selected from fluorine, hydroxy, and C₁₋₃ alkoxy,
C₂₋₆ alkenyl, wherein alkenyl is optionally substituted with one to five substituents independently selected from fluorine, hydroxy, and C₁₋₃ alkoxy,
(CH₂)ₙ-aryl,
(CH₂)ₙ-heteroaryl,
(CH₂)ₙ-heterocyclyl,
(CH₂)ₙ—C₃₋₆ cycloalkyl,
(CH₂)ₙ—OR⁹,
(CH₂)ₙ—CO₂R⁹,
(CH₂)ₙ—N(R⁹)₂,
(CH₂)ₙ—CON(R⁹)₂,
(CH₂)ₙ—OCON(R⁹)₂,
(CH₂)ₙ—SO₂N(R⁹)₂,
(CH₂)ₙ—SO₂N(R⁹)C(O)R⁹,
(CH₂)ₙ—C(O)N(R⁹)SO₂R¹⁰,
(CH₂)ₙ—S(O)ᵣR¹⁰,
(CH₂)ₙ—NR¹¹SO₂R¹⁰,
(CH₂)ₙ—NR¹¹CON(R⁹)₂,
(CH₂)ₙ—NR¹¹COR⁹, and
(CH₂)ₙ—NR¹¹CO₂R¹⁰;
wherein aryl, heteroaryl, cycloalkyl, and heterocyclyl are optionally substituted with one to three substituents independently selected from halogen, hydroxy, C₁₋₄ alkyl, trifluoromethyl, and C₁₋₄ alkoxy; and wherein any methylene (CH₂) carbon atom in R^{c} is optionally substituted with one to two groups independently selected from fluorine, hydroxy, and C₁₋₄ alkyl optionally substituted with one to three fluorines; or two substituents when on the same methylene (CH₂) group are taken together with the carbon atom to which they are attached to form a cyclopropyl group;
R⁴, R⁵, R⁷, and R⁸ are each independently selected from the group consisting of:
hydrogen,
halogen,
C₁₋₄ alkyl, optionally substituted with one to five fluorines,
C₁₋₄ alkoxy, optionally substituted with one to five fluorines, and
C₁₋₄ alkylthio, optionally substituted with one to five fluorines;
R⁶ is selected from the group consisting of:
—(CH₂)ₘ-aryl,
—(CH₂)ₘ-heteroaryl,
—OCH₂-aryl,
—OCH₂-heteroaryl,
—(S)ᵣCH₂-aryl,
—(S)ᵣCH₂-heteroaryl,
—CH₂O-aryl,
—CH₂O-heteroaryl,
—CH₂(S)ᵣ-aryl, and
—CH₂(S)ᵣ-heteroaryl;
wherein any methylene (CH₂) carbon atom in R⁶ is optionally substituted with one to two groups independently selected from fluorine, hydroxy, and C₁₋₄ alkyl optionally substituted with one to three fluorines; or two substituents when on the same methylene (CH₂) group are taken together with the carbon atom to which they are attached to form a cyclopropyl group and wherein aryl and heteroaryl are optionally substituted with one to three R^{d} substituents independently selected from the group consisting of:
halogen,
cyano,
C₁₋₄ alkyl, optionally substituted with one to five fluorines,
C₁₋₄ alkoxy, optionally substituted with one to five fluorines,
C₁₋₄ alkylthio, optionally substituted with one to five fluorines, and
C₁₋₄ alkylsulfonyl, optionally substituted with one to five fluorines;
each R⁹ is independently selected from the group consisting of
hydrogen,
C₁₋₆ alkyl,
(CH₂)ₘ-aryl,
(CH₂)ₘ-heteroaryl, and
(CH₂)ₘC₃₋₆ cycloalkyl;
wherein any individual methylene (CH₂) carbon atom in (CH₂)ₘ is optionally substituted with one to two substituents independently selected from fluorine, hydroxy, C₁₋₄ alkyl, and C₁₋₄ alkoxy, wherein alkyl and alkoxy are optionally substituted with one to five fluorines; or two substituents when on the same methylene (CH₂) group are taken together with the carbon atom to which they are attached to form a cyclopropyl group; and wherein alkyl, aryl, heteroaryl, and cycloalkyl are optionally substituted with one to three substituents independently selected from the group consisting of halogen, C₁₋₄ alkyl, and C₁₋₄ alkoxy; or two R⁹ groups substituents together with the nitrogen atom to which they are attached form a heterocyclic ring selected from azetidine, pyrrolidine, piperidine, piperazine, and morpholine wherein said heterocyclic ring is optionally substituted with one to three substituents independently selected from the group consisting of halogen, hydroxy, C₁₋₆ alkyl, and C₁₋₆ alkoxy, wherein alkyl and alkoxy are optionally substituted with one to five fluorines;
each R¹⁰ is independently C₁₋₆ alkyl, wherein alkyl is optionally substituted with one to five substituents independently selected from fluorine and hydroxy;
R¹¹ is hydrogen or R¹⁰;
each n is independently an integer from 0 to 3;
each m is independently an integer from 0 to 2; and
each r is an integer from 0 to 2.

2. The compound of claim 1 wherein R³ and R⁶ are each independently aryl or heteroaryl wherein R³ is optionally substituted with one to three R^{c} substituents, and R⁶ is optionally substituted with one to three R^{d} substituents.

3. The compound of claim 2 wherein R³ is phenyl or thienyl each of which is optionally substituted with one to three R^{c} substituents.

4. The compound of claim 3 wherein R³ is 3-thienyl optionally substituted with one to two R^{c} substituents.

5. The compound of claim 2 wherein R⁶ is phenyl or pyridyl each of which is optionally substituted with one to three R^{c} substituents.

6. The compound of claim 1 wherein R³ is aryl or heteroaryl wherein aryl and heteroaryl are optionally substituted with one to three R^{c} substituents; and R⁶ is —OCH₂-aryl or —OCH₂-heteroaryl wherein aryl and heteroaryl are optionally substituted with one to three R^{d} substituents.

7. The compound of claim 6 wherein R³ is phenyl or thienyl wherein phenyl and thienyl are optionally substituted with one to three R^{c} substituents; and R⁶ is —OCH₂-phenyl or —OCH₂-pyridyl wherein phenyl and pyridyl are optionally substituted with one to three R^{d} substituents.

8. The compound of claim 7 wherein R³ is 3-thienyl optionally substituted with one to two R^{c} substituents.

9. The compound of claim 1 wherein R⁶ is aryl or heteroaryl wherein aryl and heteroaryl are optionally substituted with one to three R^{d} substituents; and R³ is —OCH₂-aryl or —OCH₂-heteroaryl wherein aryl and heteroaryl are optionally substituted with one to three R^{c} substituents.

10. The compound of claim 9 wherein R⁶ is phenyl optionally substituted with one to three R^{c} substituents; and R³ is —OCH₂-phenyl or —OCH₂-pyridyl wherein phenyl and pyridyl are optionally substituted with one to three R^{d} substituents.

11. The compound of claim 10 wherein R³ is —OCH₂-aryl or —OCH₂-heteroaryl wherein aryl and heteroaryl are optionally substituted with one to three R^{c} substituents; and R⁶ is —OCH₂-aryl or —OCH₂-heteroaryl wherein aryl and heteroaryl are optionally substituted with one to three R^{d} substituents as defined above.

12. The compound of claim 11 wherein R³ is —OCH₂-phenyl or —OCH₂-pyridyl wherein phenyl and pyridyl are optionally substituted with one to three R^{c} substituents; and R⁶ is —OCH₂-phenyl wherein phenyl is optionally substituted with one to three R^{d} substituents.

13. The compound of claim 1 wherein R² is fluoro or hydrogen.

14. The compound of claim 1 wherein R¹ is hydrogen.

15. The compound of claim 14 wherein R² is fluoro or hydrogen, and R⁴, R⁵, R⁷, and R⁸ are each hydrogen.

16. The compound of claim 1 wherein R^{d} is selected from the group consisting of:
halogen,
C₁₋₃ alkyl, optionally substituted with one to three fluorines,
C₁₋₃ alkoxy, optionally substituted with one to three fluorines, and
C₁₋₃ alkylthio, optionally substituted with one to three fluorines.

17. The compound of claim 1 wherein R^{c} is selected from the group consisting of:
C₁₋₃ alkoxy, optionally substituted with one to three fluorines,
—CO₂R⁹,
—S(O)ᵣR¹⁰,
—C(O)R⁹. 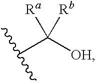
heterocyclyl, and
heteroaryl;
and R^{a} and R^{b} are each independently hydrogen or methyl, wherein methyl is optionally substituted with one to three fluorines.

18. The compound of claim 17 wherein R^{c} is selected from the group consisting of:

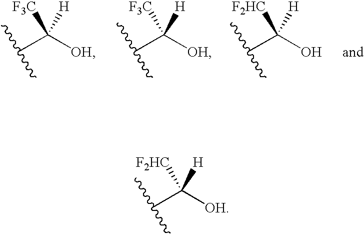

19. The compound of claim 17 wherein R^{c} is heteroaryl or heterocyclyl wherein heteroaryl and heterocyclyl are optionally substituted with one to two substituents independently selected from halogen, hydroxy, C₁₋₄ alkyl, trifluoromethyl, and C₁₋₄ alkoxy.

20. The compound of claim 1 wherein R³ is phenyl monosubstituted at the para position with an R^{c} substituent.

21. The compound of claim 1 wherein R⁶ is phenyl monosubstituted at the para position with an R^{d} substituent.

22. A pharmaceutical composition comprising a compound in accordance with claim 1 in combination with a pharmaceutically acceptable carrier.

23-25. (canceled)

26. A method for treating non-insulin dependent (Type 2) diabetes, insulin resistance, hyperglycemia, a lipid disorder, obesity, and conditions associated with the Metabolic Syndrome in a mammal in need thereof which comprises the administration to the mammal of a therapeutically effective amount of a compound of claim 1.
